(19) United States
(12) Patent Application Publication
(10) Pub. No.: US 2024/0099805 A1
(43) Pub. Date: Mar. 28, 2024

(54) METHOD AND APPARATUS FOR GUIDING A SURGICAL ACCESS DEVICE

(71) Applicant: Medos International Sarl, Le Locle (CH)

(72) Inventors: Joern Richter, Kandern (DE); Thomas Gamache, Westport, MA (US); John Riley Hawkins, Cumberland, RI (US)

(21) Appl. No.: 17/935,192

(22) Filed: Sep. 26, 2022

(51) Int. Cl. A61B 90/00; A61B 34/20

(52) U.S. Cl. CPC A61B 90/361; A61B 34/20; A61B 2034/107

(57) ABSTRACT

A system for facilitating a medical procedure includes an access device that is configured to provide distance information regarding anatomical structures as the access device is driven to a target anatomical site. For instance, the access device can have either or both of a camera and neuromonitoring electrodes. The camera can send real-time images of the anatomical structures as the access device is driven toward the target anatomical site. The electrodes can emit an electrical current to identify a distance from nerve structure. A visual display can include the anatomical structure identified to the operator to assist in guiding the access device to the target anatomical site.

### TECHNICAL FIELD

The present disclosure is generally related to systems and methods for facilitating implantation of an anatomical implant at a target surgical location, and in particular relates to systems and methods for facilitating implantation of an intervertebral implant into an intervertebral space through Kambin's triangle.

### BACKGROUND

Perioperative neurological injury is a known complication associated with elective spinal surgery. Neurological injury can result when contact occurs with neurological structures during a surgical procedure. Some examples of perioperative neurological complications that may result from spinal surgery include vascular injury, durotomy, nerve root injury, and direct mechanical compression of the spinal cord or nerve roots during vertebral column instrumentation. Wide variation in patient anatomy can make it difficult to accurately predict or identify a location of neurological structures in a particular patient's spinal region.

According to data from the National Institute of Science, the incidence of perioperative neurological injuries resulting from elective spine surgery increased 54.4%, from 0.68% to 1%, between 1999 and 2011. Additionally, perioperative neurological complications in elective spine surgery were associated with longer hospital stays (9.68 days vs. 2.59 days), higher total charges ($110,326.23 vs. $48,695.93), and increased in-hospital mortality (2.84% vs. 0.13%).

While minimally invasive spine surgery (MISS) has many known benefits, multi-study analysis of patient outcome data for lumbar spine surgery indicate that MISS has a significantly higher rate of nerve root injury (2%-23.8%) as compared to traditional ‘open’ surgical techniques (0%-2%). With MISS procedures, accessing the spine or a target surgical location often involves navigating a surgical instrument through patient anatomy including muscles, fatty tissue, and neurological structures. Current intra-operative imaging devices do not adequately show neurological structures in an operating region. For example, computed tomography (CT) and cone beam computed tomography (CBCT) imaging technology is often used intra-operatively to visualize musculoskeletal structures in an operating region of a patient's anatomy. CT and CBCT images, however, do not show neurological structures. Furthermore, the current practice is to use CT imaging for preoperative planning of a surgical approach. Since neurological structures are not visible in CT image volumes, a surgical approach cannot be optimized to avoid or reduce contact with neurological structures. While magnetic resonance imaging (MM) imaging shows both musculoskeletal and neurological structures of a scanned patient anatomy, Mill imaging is typically used only to diagnose a patient and not for pre-operative surgical planning or intra-operative use.

Although the incidence of perioperative neurological injury in MISS procedures is greater than traditional open surgical techniques, MISS remains an attractive treatment option for spinal disorders requiring surgery. Benefits of MISS, as compared to open surgery, include lower recovery time, less post-operative pain, and smaller incisions.

Accordingly, there is a need for systems and methods for reducing the incidence of neurological complications in spinal surgery, and, in particular, for reducing the incidence of neurological complications in minimally invasive spinal surgery.

During MISS surgery, it is difficult to identify anatomical structures, even for experienced practitioners, and therefore multiple technologies are often utilized. CT non-invasive scans comprise of X-rays to produce detailed, three-dimensional (3D) images of a region of interest (ROI) of a body or patient (e.g., person or animal). Mill comprises non-invasive use of magnets to create a strong magnetic field and pulses to create 3D images of the target or region of interest. Endoscopes provide visual information in real-time on the surgery site. CT and Mill scans can be overlaid on a camera's image and visualizations may be performed via augmented reality (AR) or virtual reality (VR).

### SUMMARY

In one example, a surgical system includes a trocar having a trocar body and first and second sensors supported by the trocar body, wherein each of the first and second sensors is configured to sense at least one of a position of the trocar, an orientation of the trocar, a property of tissue proximate to the trocar, and a distance from a tissue and the trocar. The surgical system can further include a display, and a in communication with the plurality of sensors and the display. The processor can be configured to overlay on the display graphical representations of data from each of the sensors as the trocar is advanced toward a target anatomical site.

### BRIEF DESCRIPTION OF THE DRAWINGS

The details of particular implementations are set forth in the accompanying drawings and description below. Like reference numerals may refer to like elements throughout the specification. Other features will be apparent from the following description, including the drawings and claims. The drawings, though, are for the purposes of illustration and description only and are not intended as a definition of the limits of the disclosure.

FIG. 1A is a lateral elevation view of a portion of a vertebral column;

FIG. 1B is a posterior elevation view of the vertebral column of FIG. 1A;

FIG. 1C is a plan view of a vertebra of the vertebral column of FIG. 1B;

FIG. 2 is a schematic side view of Kambin's triangle;

FIG. 3A is a perspective view of a trocar constructed in accordance with one example;

FIG. 3B is an enlarged side elevation view of a portion of the trocar of FIG. 3A;

FIG. 3C is an enlarged exploded side elevation view of a portion of the trocar of FIG. 3A;

FIG. 3D is an enlarged side elevation view of a portion of the trocar of FIG. 3A in another example;

FIG. 3E is an enlarged side elevation view of a portion of the trocar of FIG. 3A in yet another example;

FIG. 3F is an enlarged perspective view of a portion of the trocar of FIG. 3A in yet another example;

FIG. 4A is a preoperative image of anatomical structures of a patient prior to a surgical procedure through Kambin's triangle;

FIG. 4B is a real-time image from a camera of the trocar of FIG. 3A overlaid by the image of FIG. 4A;

FIG. 5A is a perspective view of an access assembly including the trocar of FIG. 3A and a flexible surgical access port removably coupled to the trocar;

FIG. 5B is another perspective view of the flexible surgical access port;

FIG. 6A is a perspective view of the flexible surgical access port of FIG. 3 constructed in accordance with one example;

FIG. 6B is a perspective view of the flexible surgical access port of FIG. 6A conforming to an irregular shape;

FIG. 6C is a perspective view of the flexible surgical access port of FIG. 6A conforming to an alternative irregular shape;

FIG. 7 is a perspective view of one embodiment of a spinal fusion cage aligned for insertion through the flexible distal access port of FIG. 5B and into the intervertebral space;

FIG. 8A illustrates a surgical system for assisting minimally invasive spine surgery in accordance with one or more embodiments;

FIG. 8B further illustrates an example of the surgical system of FIG. 8A;

FIG. 9 illustrates a process for improving accuracy of a medical procedure, in accordance with one or more embodiments; and

FIG. 10 illustrates a process for facilitating a situationally aware medical procedure, in accordance with one or more embodiments.

### DETAILED DESCRIPTION

Certain embodiments disclosed herein are discussed in the context of an intervertebral implant and spinal fusion because of the device and methods have applicability and usefulness in such a field. The device can be used for fusion, for example, by inserting an intervertebral implant to properly space adjacent vertebrae in situations where a disc has ruptured or otherwise been damaged. “Adjacent” vertebrae can include those vertebrae originally separated only by a disc or those that are separated by intermediate vertebra and discs. Such embodiments can therefore be used to create proper disc height and spinal curvature as required in order to restore normal anatomical locations and distances. However, it is contemplated that the teachings and embodiments disclosed herein can be beneficially implemented in a variety of other operational settings, for spinal surgery and otherwise.

As used throughout this application, the word “may” is used in a permissive sense (i.e., meaning having the potential to), rather than the mandatory sense (i.e., meaning must). The words “include,” “including,” and “includes” and the like mean including, but not limited to. As used herein, the singular form of “a,” “an,” and “the” include plural references unless the context clearly dictates otherwise. As employed herein, the term “number” shall mean one or an integer greater than one (i.e., a plurality).

As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

These drawings may not be drawn to scale and may not precisely reflect structure or performance characteristics of any given embodiment, and should not be interpreted as defining or limiting the range of values or properties encompassed by example embodiments.

Unless specifically stated otherwise, as apparent from the discussion, it is appreciated that throughout this specification discussions utilizing terms such as “processing,” “computing,” “calculating,” “determining,” or the like refer to actions or processes of a specific apparatus, such as a special purpose computer or a similar special purpose electronic processing/computing device.

Certain exemplary embodiments will now be described to provide an overall understanding of the principles of the structure, function, manufacture, and use of the devices, systems, and methods disclosed herein. One or more examples of these embodiments are illustrated in the accompanying drawings. Those skilled in the art will understand that the devices, systems, and methods specifically described herein and illustrated in the accompanying drawings are non-limiting exemplary embodiments. The features illustrated or described in connection with one exemplary embodiment may be combined with the features of other embodiments. Such modifications and variations are intended to be included within the scope of the present disclosure.

Additionally, to the extent that linear or circular dimensions are used in the description of the disclosed devices and methods, such dimensions are not intended to limit the types of shapes that can be used in conjunction with such devices and methods. A person skilled in the art will recognize that an equivalent to such linear and circular dimensions can easily be determined for any geometric shape. Still further, sizes and shapes of the devices, and the components thereof, can depend at least on the anatomy of the subject in which the devices will be used, the size and shape of components with which the devices will be used, and the methods and procedures in which the devices will be used.

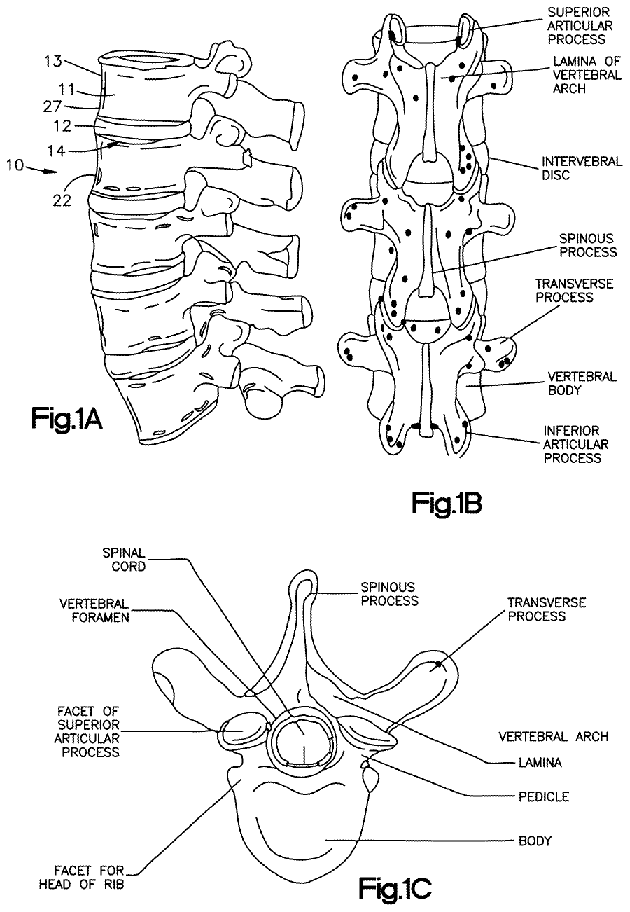

As context for the methods and devices described herein, FIGS. 1A-1C are different views of a vertebral column 10 including a series of alternating vertebrae 11 having vertebral bodies 13, and intervertebral disc spaces 14 that contain fibrous intervertebral discs that provide axial support and movement to the upper portions of the body. The vertebral column typically comprises thirty-three vertebrae 11, with seven cervical (C1-C7), twelve thoracic (T1-T12), five lumbar (L1-L5), five fused sacral (S1-S5), and four fused coccygeal vertebrae. Discs 12 are disposed in intervertebral spaces between adjacent vertebral bodies, and provide axial support and movement to the upper portions of the body. At times it becomes desirable to remove the disc 12 and insert an intervertebral implant into the disc space to restore height and alignment of the vertebrae.

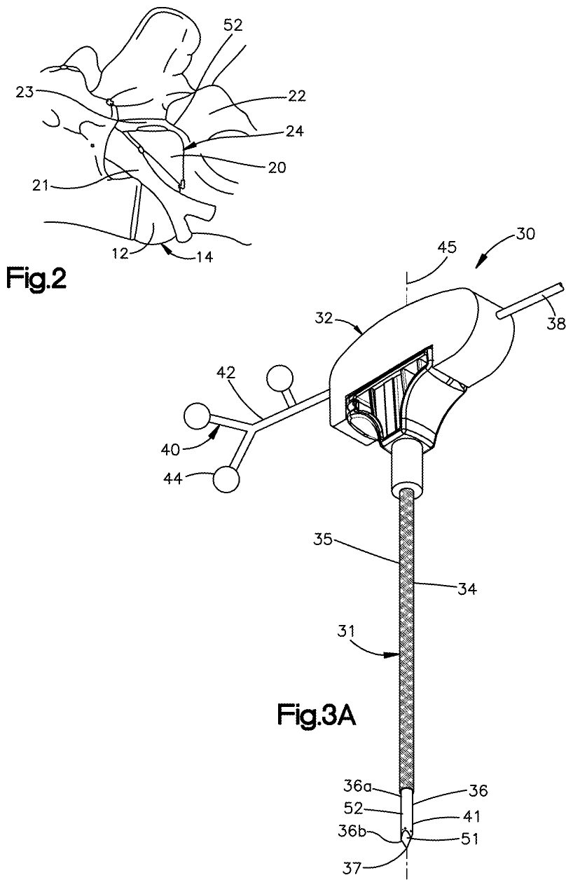

FIG. 2 is a schematic view of Kambin's triangle 24. This region 20 is the site of posterolateral access for spinal surgery. It can be defined as a right triangle over the intervertebral disc 12 viewed dorsolaterally. The hypotenuse is the exiting nerve 21, the base is the superior border of the inferior vertebra 22, and the height is the traversing nerve root 23. In some examples, the intervertebral disc space 14, and thus the disc 12, can be accessed by performing a foraminoplasty in which a portion of the superior articular process (SAP) 53 of the inferior vertebra 22 is removed such that surgical equipment, such as surgical instruments or implants can be introduced through the Kambin's triangle 24. The intervertebral disc space 14 is defined by the vertebrae of the inferior vertebra 22 and a superior vertebra 27 that is opposite the inferior vertebra 22. The portion of the inferior vertebra 22 that is removed can be defined by the superior articular process of the inferior vertebra 22. In such a procedure, it is often desired to protect the exiting nerve and the traversing nerve root. Apparatus and methods for accessing the intervertebral disc through Kambin's triangle 24 may involve performing endoscopic foraminoplasty while protecting the nerve will be discussed in more detail below. Utilizing foraminoplasty to access the intervertebral disc 12 through Kambin's triangle 24 can have several advantages (e.g., less or reduced trauma to the patient) as compared to accessing the intervertebral disc posteriorly or anteriorly as is typically done in the art.

In particular, surgical procedures involving posterior access often require removal of the facet joint. For example, transforaminal interbody lumbar fusion (TLIF) typically involves removal of one facet joint to create an expanded access path to the intervertebral disc. Removal of the facet joint can be very painful for the patient, and is associated with increased recovery time. In contrast, accessing the intervertebral disc through Kambin's triangle 24 may advantageously avoid the need to remove the facet joint.

As described in more detail below, endoscopic foraminoplasty may provide for expanded access to the intervertebral disc without removal of a facet joint. Sparing the facet joint may reduce patient pain and blood loss associated with the surgical procedure. In addition, sparing the facet joint can advantageously permit the use of certain posterior fixation devices which utilize the facet joint for support (e.g., trans-facet screws, trans-pedicle screws, and/or pedicle screws). In this manner, such posterior fixation devices can be used in combination with interbody devices inserted through the Kambin's triangle 24.

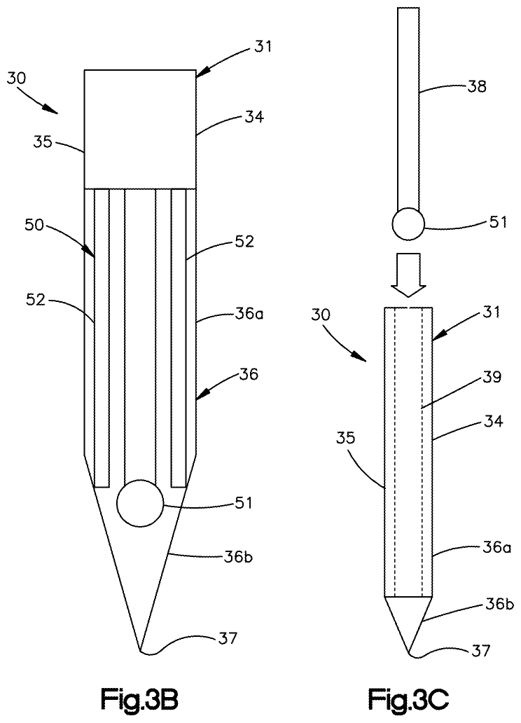

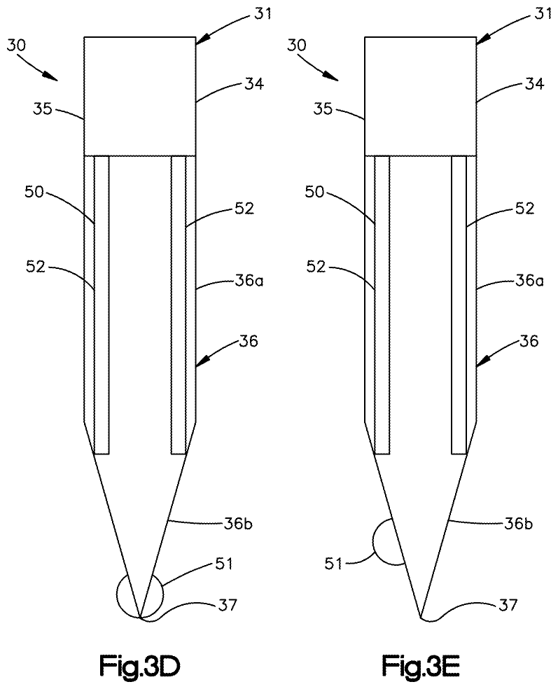

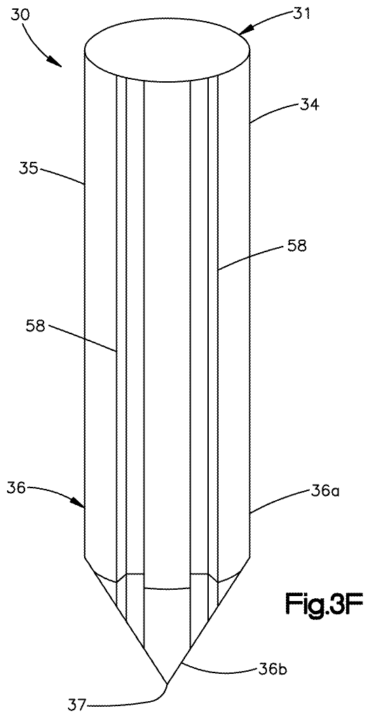

As will now be described with reference to FIGS. 3A-3E, a surgical system 25 can include a trocar 30 that is configured to be driven through anatomical tissue along a trajectory toward Kambin's triangle 24 so as to create a path to Kambin's triangle. The trocar 30 can be further driven along the trajectory through Kambin's triangle 24 to a target surgical location which can be defined by a desired intervertebral disc space or vertebra. While reference is made herein to a trocar, it is appreciated that the embodiments disclosed herein can be used with any access device that is configured to be driven through anatomical tissue along a trajectory toward a target anatomical site so as to create a path to the target anatomical site. The access device can be further driven along the trajectory through the target anatomical site to a target surgical location. In one example, the access device can be defined by the trocar 30, the target anatomical site can be defined by Kambin's triangle, and the target surgical location can be defined by an intervertebral disc space. The target surgical location can also be referred to as a target anatomical site. Further, in some examples, for instance where a portion of the superior articular process is removed to enlarge Kambin's triangle, Kambin's triangle can be referred to as a target surgical location.

The trocar 30 can include a trocar body 31 and a camera 51 supported by the trocar body 31. The camera 51 can be an optical camera that obtains images that can be displayed to the user or manipulated by the surgical system to be overlayed in a composite image as described in more detail below. Alternatively or additionally, the camera 51 can be hyper-spectral to identify and differentiate different tissue types (e.g., nerve, musculature, bone, arteries, and the like) in response to different types of light that can be emitted by the trocar 30 and in particular by the camera 51. That is, it is recognized that different tissue types respond differently to different types of light in a hyperspectral light range. Therefore, the trocar 30, and in particular the camera 51, or any suitable alternative light source can direct the light in the hyperspectral range to the tissue to be identified. An identification of the tissue type can be determined based on the response of the tissue to a particular light within the hyperspectral range.

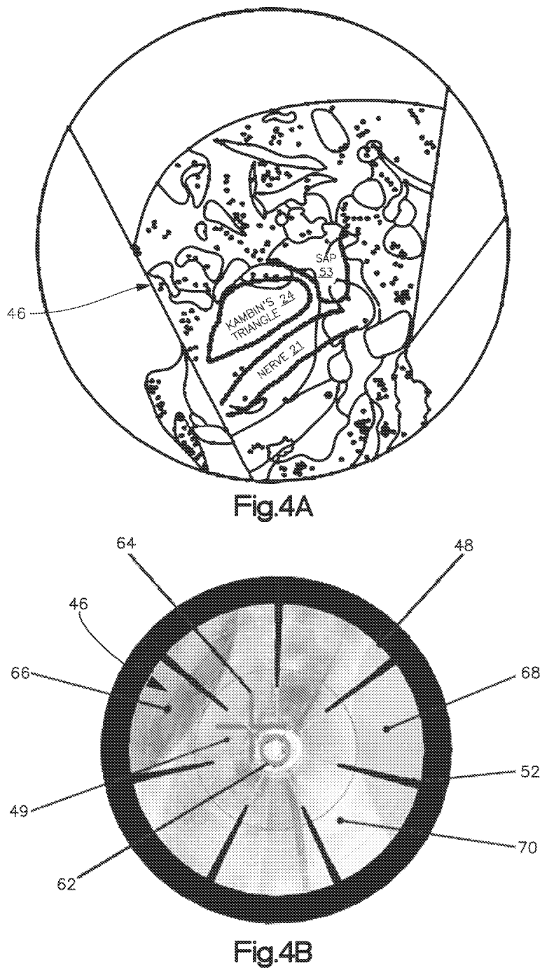

It is therefore appreciated that the camera 51 can be referred to as a non-visible light image sensor, such that the processor can differentiate tissue type (for instance between bone, soft tissue, and nerve) based on images captured by the non-visible light image sensor. Alternatively or additionally, the camera 51 can be configured as a visible light sensor. As is described in more detail below, the trocar 30 is driven into the anatomy toward Kambin's triangle, the processor can receive the images from the camera 51 and can identify the structure in the field of view of the camera based on either or both of the known location of the camera 51 and pre-operative images taken of the patient anatomy prior to the surgical procedure (see FIG. 4A). One or more of the pre-operative images can be overlaid onto the real-time images of the camera as shown in FIG. 4B and can provide an identification of the anatomical structure in the field of view of the camera 51.

The trocar body 31 can be made of any suitable biocompatible material(s). The trocar body 31 can include a trocar handle 32 and a trocar shaft 34 that extends from the trocar handle 32 along a central axis 45 of the trocar 30. The central axis of the trocar 30 can define a central axis of the trocar shaft 34. The trocar shaft 34 can be solid or cannulated as desired. The trocar handle 32 can be enlarged with respect to the trocar shaft 34 in a cross-section perpendicular to the distal direction. The trocar shaft 34 can have main shaft portion 35 and a distal region 36 that extends from the main shaft portion 35. The distal region 36 can be made from a different material than the main shaft portion 35. Alternatively, the distal region 36 can be made from the same material as the main shaft portion 35. The distal region 36 can terminate at a distal tip 37 that defines the distal end of both the trocar shaft 34 and the trocar body 31, and thus also of the trocar 30. The distal end can define a leading end with respect to insertion through anatomical tissue toward the target intervertebral space through Kambin's triangle.

The distal tip 37 can be disposed on the central axis of the trocar 30. The trocar handle 32 can define a proximal end of the trocar body 31. A distal direction can be defined as a direction from the proximal end to the distal end of the trocar body 31. Thus, the distal region 36 can extend in the distal direction with respect to the main shaft portion 35. For instance, the distal region 36 can extend from the main shaft portion 35 in the distal direction. A proximal direction opposite the distal direction can be defined as a direction from the distal end of the trocar 30 to the proximal end of the trocar body 31. The proximal and distal directions can each be defined by the central axis of the trocar 30, which can be oriented along a longitudinal direction L.

At least a portion of the distal region 36 of the trocar shaft 34 can taper to the distal tip 37 as it extends in the distal direction. For instance, a first portion 36a of the distal region 36 can extend along the longitudinal direction L without tapering. A second portion 36b of the distal region 36 can taper to the distal tip 37 as it extends in the distal direction. The second portion 36b of the distal region 36 can extend in the distal direction from the first portion 36a of the distal region 36. The first portion 36a of the distal region 36 can extend in the distal direction from the main shaft portion 35. In other examples, an entirety of the distal region 36 can taper from the main shaft portion 35 to the distal tip 37. That is, the entirety of the distal region 36 can be defined as described with respect to the second portion 36b that extends from the main shaft portion 35.

In one example, the second portion 36b of the distal region 36 can be conical. In other examples the second portion 36b of the distal region 36 can be define one or more flat surfaces as it extends in the distal direction. The flat surfaces can be adjacent each other so as to define a structure that extends about the central axis of the trocar shaft 34. For instance, the second portion 36b of the distal region 36 can be pyramidal, such as a rectangular pyramid, triangular pyramid, hexagonal pyramid, or the like. Thus, the distal region 36 can have at least one side wall 41 that is tapered. In some example, the tapered side wall 41 can comprise a plurality of tapered side walls 41. The distal tip 37 can be a sharp distal tip that defines, for instance, an apex of a cone or a vertex of a pyramid. Thus, the second portion 36b of the distal region 36 can be conical. In other examples, the distal tip 37 can be blunt. Thus, the second portion 36b of the distal region 36 can be frustoconical in one example. The sharp distal tip can be advantageous when penetrating fascia and soft tissue as the trocar is inserted toward a target surgical location. The target surgical location can be defined, for instance, by an intervertebral space that is accessed through Kambin's triangle.

Thus, during operation, the trocar 30 can be driven along a desired trajectory through Kambin's triangle to the intervertebral space without contacting the exiting nerve 21 or the traversing nerve root 23. As the trocar 30 can be the first instrument inserted to or through Kambin's triangle during the surgical procedure, the trocar 30 can establish the trajectory through Kambin's triangle. In some examples, the trocar 30 can be driven in the distal direction along the desired trajectory through Kambin's triangle.

The surgical system 25 can be configured to inform the operator whether the trocar 30 is being driven along a trajectory to an intervertebral disc space through Kambin's triangle while avoiding contact with the surrounding nerves and other tissue. In particular, the camera 51 can have direct real-time visualization of anatomical structure in the field of view of the camera 51 as the trocar 30 is driven to the intervertebral disc space through Kambin's triangle. The camera 51 can provide real-time images of the anatomical tissue in its field of view as the trocar 30 is driven toward Kambin's triangle 24. The surgical system 25 can determine the identity of the tissue in the field of view of the camera 51, and can determine whether the trocar 30 is on the trajectory through Kambin's triangle. In particular, as described in more detail below, the images of the anatomical tissue from the camera 51 can be provided on a display. In some examples, the surgical system 25 can overlay the camera images onto one or more pre-operative images of the patient's anatomical structure, such that the anatomical structures of the camera images achieve a fit with like anatomical structures of the one or more pre-operative images. The pre-operative images can resemble that of FIG. 2. Thus, the surgical system 25 can identify on the overlaid images the anatomical structure in the field of view of the camera 51 that is relevant to the approach of the trocar 30 to or through Kambin's triangle 24, including the exiting nerve 21, the SAP 53, and the like (see FIG. 4A). The images can further include the traversing nerve root 23 in some examples (see FIG. 2).

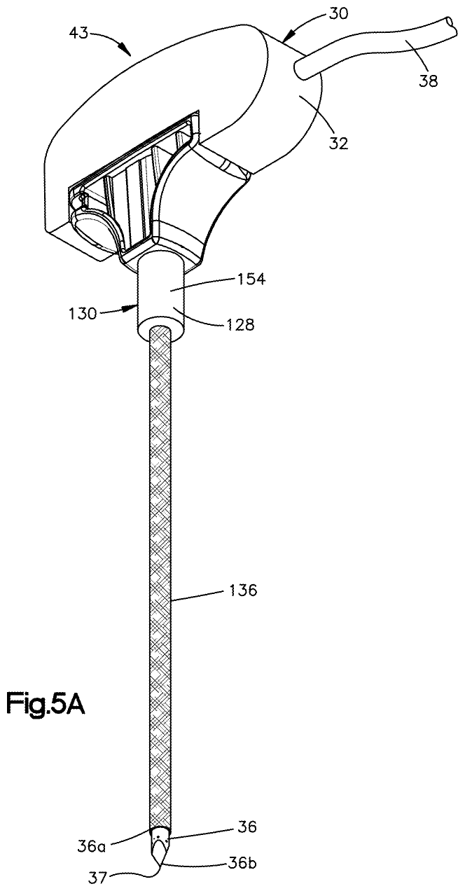

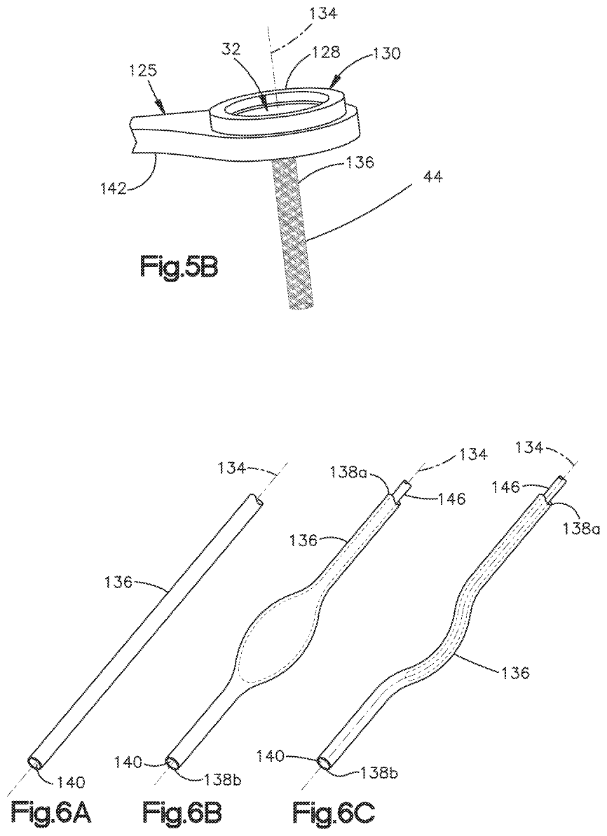

The surgical system 25 can further provide feedback to the operator if it is determined that the actual trajectory of the trocar 30 is different than the desired trajectory or outside a tolerance value of the desired trajectory (collectively referred to as substantially different than the desired trajectory). The feedback can be a visual indicator. Alternatively or additionally, the feedback can be a haptic feedback. For instance, the trocar handle 32 can include a vibrating actuator 55 (see FIG. 5A) when the actual trajectory of the trocar 30 is substantially different than the desired trajectory. Alternatively or additionally still, the feedback can be an audible feedback along with correction instructions to change the actual trajectory to the desired trajectory. Any of the feedback described above can also inform the operator that the actual trajectory of the trocar 30 is the desired trajectory or within tolerance of the desired trajectory.

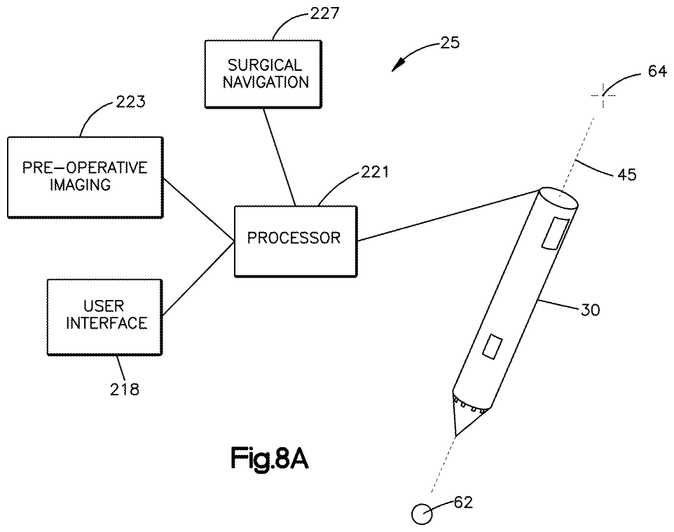

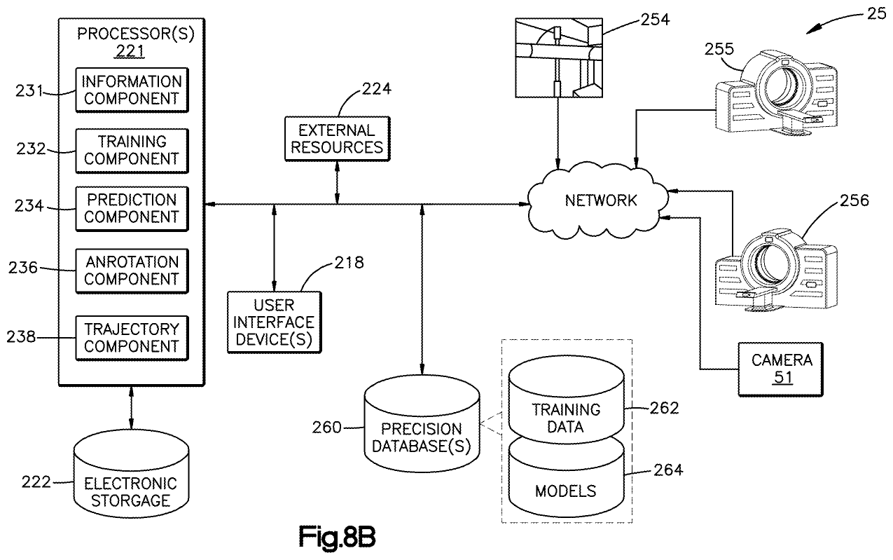

In particular, the camera 51 can be in communication with a processor 221 (see FIGS. 8A-8B) and/or can be stored in memory in any manner as desired. For instance, a communication cable 38 can extend from the camera 51 to the computing device that includes processor 221 and/or memory to facilitate the exchange of data to and/or from the camera 51 and the processor 221. For instance, image data can be communicated from the camera 51, and imaging instructions can be communicated to the camera 51. The communication cable 38 can extend out the trocar handle 32. Alternatively, the camera 51 can be in wireless communication with the processor 221 and/or memory.

The camera 51 can be supported by the trocar body 31 in any suitable manner as desired. For instance, referring to FIGS. 2-3C, the trocar body 31 can define a lumen 39. The lumen 39 can extend into or through the trocar shaft 34. Alternatively or additionally, the lumen 39 can extend into or through the distal region 36. The lumen 39 can be a central lumen, such that the central axis of the trocar body 31 extends through the lumen 39. The camera 51 can be disposed in the lumen 39, and can be supported by either or both of the trocar shaft 34 and the distal region 36. Thus, the camera 51 can be disposed on an interior surface of the trocar body 31 that faces or partially defines the lumen 39.

The camera 51 can be centrally located on the central axis of the trocar 30, and the field of view of the camera can be directed distally and centered substantially about the central axis. At least a portion of the distal region 36 can be transparent, such that the field of view of the camera 51 extends through the distal region 36. The distal region 36 can be made of any suitable material such as plastic or glass. While a sharp distal tip 37 can assist with penetration of soft tissue as the trocar is driven toward the target intervertebral space through Kambin's triangle, a blunt distal tip 37 can offer better viewing of the camera 51 through the distal tip 37. It should be appreciated, of course, that an entirety of the distal region 36 can be transparent. The field of view of the camera 51 can be centered with respect to the central axis of the trocar 30 and include data from tissue around and adjacent to distal region 36. Further, the viewing angle of the camera 51 can be oriented in the distal direction, which can define the direction of insertion of the trocar 30 toward Kambin's triangle. The communication cable 38 can extend proximally through the lumen 39 and out the trocar handle 32. Further, at least a portion of the distal region 36 can carry a light source that can be activated to illuminate the field of view of the camera 51. The light source control signals can be communicated over the communication cable 38. The light source can be in the lumen 39 or attached to an exterior surface of the trocar 30 as desired.

In addition to the visual indicia sent from the camera 51 to the processor 221 and/or memory (see FIGS. 8A-8B), the trocar 30 can also provide apparatus that facilitates a determination of the depth of insertion of the trocar 30 as it is driven toward Kambin's triangle. In one example, the trocar 30 can include a reference array 40 that can include a reference array body 42 and a plurality of markers 44 supported by the reference array body 42. The reference array body 42 can be attached to any suitable location of the trocar body 31. In one example, the reference array body 42 extends from the trocar handle 32. The markers 44 can be radio-opaque in some examples. The markers 44 can be positionally fixed relative to the reference array body 42 when the markers 44 are coupled to the reference array body 42, such that movement of the reference body 42 causes corresponding movement of the markers 44. Each marker 104 can have any suitable shape as desired, such as spherical or partially spherical. It should be appreciated that the reference array 40, and in particular the markers 44, provide a sensor that determines a location and orientation of the trocar 30.

The markers 44 can be detected by the processor 221 and/or memory (see FIGS. 8A-8B) so that the position and orientation of the trocar 30 (and thus movement of the trocar 30) can be determined. In particular, the position of each of the markers 44 can be determined prior to driving the trocar 30 toward Kambin's triangle (or at some other known location). Based on subsequent detection of the position of each of the markers 44, the processor can determine the direction and distance that the trocar 30 has travelled. The processor can further determine the orientation of the trocar 30 based on the positions of the markers 44 relative to each other. As will be described in more detail below, the known position and orientation of the trocar 30 along with the images produced from the camera 51 can be processed to determine the anatomical structures in the field of view of the camera 51. The determination of the anatomical structures in the field of view of the camera 51 can be augmented by pre-operative scans of the patient's anatomy. For instance, the images from the camera 51 can be overlaid onto the pre-operative images to provide a confidence interval of the various anatomical structures (e.g., the SAP, exiting nerve 21, nerve root 23, Kambin's triangle 24, soft tissue, other bony structure, and the like). The surgical system 25 provide real-time output to the operator of an indication of any difference between the actual trajectory (taking into account changes in position and orientation of the trocar 30) relative to a desired trajectory through Kambin's triangle. The difference can include distance and direction of the trocar 30 with respect to the desired trajectory. The operator can then adjust the actual trajectory of the trocar 30 to coincide with the desired trajectory. The surgical system 25 can provide output confirming that the actual trajectory coincides with the desired trajectory when no difference exists between the actual trajectory relative to the desired trajectory.

It should be appreciated that at least one marker 44 can be detected to determine a distance and direction of travel of the trocar 30. A plurality of markers 44 can be detected to further determine an orientation of the trocar 30. Each marker 44 can be a passive marker, such as a reflective marker, that can be detected by at least one sensor or camera of the computer-assisted surgery system without actively communicating with the computer of the computer-assisted surgery system 25. Alternatively, each marker 44 can be an active marker that is configured to actively communicate with the computing device of the computer-assisted surgery system 25.

As described above, the camera 51 can be supported by the trocar body 31 at any suitable location as desired. For instance, referring now to FIG. 3D, the camera 51 can be disposed on an exterior surface of the trocar body 31 that is opposite the interior surface. In one example, the camera 51 can be disposed at the distal tip 37, and can define the leading end with respect to insertion through anatomical tissue toward the target intervertebral space through Kambin's triangle. As described above, the field of view of the camera 51 can face the distal direction and can be substantially centered with respect to the central axis of the trocar 30. That is, a two-dimensional display of the field of view of the camera 51 can be oriented in a plane that is substantially perpendicular to the central axis of the trocar 30, and perpendicular to the direction of travel of the trocar to the target intervertebral space. In this regard, it should be appreciated that the distal region 36 can be opaque as desired, particularly in instances whereby the camera is attached or otherwise secured to the external surface of the trocar body 31.

Referring now to FIG. 3E, in still another example the camera 51 can be one or both of 1) disposed at a position that is directionally offset from the central axis of the trocar 30 2) facing a direction that is angularly offset with respect to the central axis of the trocar 30 and the actual trajectory of the trocar 30. In particular, the camera 51 can be disposed on an exterior surface of the trocar body 31 at a location offset from the distal tip 37. For instance, the camera 51 can be mounted or otherwise attached to the at least one side wall 41. As described above, the at least one side wall 41 can be tapered with respect to the central axis of the trocar 30 so as to define an angle with respect to the central axis of the trocar 30. In another example, the trocar 30 can be mounted to an interior surface of the at least one side wall 41 in the lumen 39 of the trocar 30. The at least one side wall 41 can be optically transparent in this example. In both FIGS. 3D and 3E, because the camera image is not taken through the distal region 36 of the trocar body 31, the distal region 36, including the first and second portions 36a and 36b of the distal region can be opaque.

Thus, the center of the field of view of the camera 51 can be directionally offset with respect to the central axes of the trocar 30. Further, the display of the field of view can be in a plane that is angularly offset with respect to a plane that is perpendicular to the central axis of the trocar 30. The display of the field of view can also be angularly offset with respect to a plane that is perpendicular to the actual trajectory of the trocar 30. The angle can be compensated in the manner described above so that the camera image displayed is a corrected image that is from a viewpoint at the distal tip 37 directed in direction that is along the actual trajectory of the trocar 30. Thus, both the directional offset and the angular offset can be compensated by the processor 221 to produce an image that is as if the camera was oriented in the actual trajectory of the trocar 30 at a location whereby the field of view is centered with respect to the actual trajectory of the trocar 30 (see FIGS. 8A-8B). The display of the corrected image can therefore be in a plane that is perpendicular to the central axis of the trocar 30, and also perpendicular to the actual trajectory of the trocar 30.

Referring now to FIG. 3F, the trocar 30 can further include recesses 58 that extend into the exterior surface of the trocar body 31 toward the central axis of the trocar. The recesses can extend into the exterior surface and can terminate prior to extending through the interior surface of the trocar body 31. The recesses 58 can be arranged circumferentially about the trocar body 31 or in any other pattern or arrangement as desired. Further, the recesses 58 can be elongate along the longitudinal direction L as desired. It is appreciated that contact between the trocar body 31 and anatomical tissue can cause the anatomical tissue to compress as the trocar 30 is driven toward Kambin's triangle. The recesses 58 can reduce contact between the trocar 30 and the anatomical tissue, thereby consequently reducing the compression of the anatomical tissue. Because a greater amount of the anatomical tissue remains uncompressed and therefore non-deformed, the surgical system 25 can more reliably identify the tissue.

Referring again to FIGS. 3A-3B, the trocar 30 and thus the surgical system 25 can include a neuro-monitoring system 50. While the neuro-monitoring system 50 is illustrated in connection with the trocar of FIGS. 3A-3B, it should be appreciated that the neuro-monitoring system 50 can be included in any trocar as described herein. The neuro-monitoring system 50 can include at least one neuro-monitoring electrode 52 that can be supported by the trocar body 31. As will now be described, the electrode 52 provides a sensor that detects proximity to a nerve, or a distance between the electrode 52 and the nerve. Because the position of the electrode 52 relative to other locations of the trocar 30 is known, the distance between any location of the trocar 30, such as the distal tip 37, and the nerve can be determined. In one example, the electrode 52 can be disposed at the distal region 36. At least a portion up to an entirety of the at least one electrode 52 can be embedded in the trocar body 31. For instance, the at least one electrode can be overmolded by the distal region 36 of the trocar body 31. Alternatively or additionally, at least a portion up to an entirety of the at least one electrode 52 can extend about the trocar body 31, for instance at the distal end 36. In one example, the at least one electrode 52 can extend along the interior surface of the trocar body 31. Alternatively, the at least one electrode 52 can extend along the exterior surface of the trocar body 31. The at least one electrode 52 can be adhesively or otherwise attached in any manner to the trocar body.

The at least one electrode 52 can in particular be supported at either or both of the first portion 36a and the second portion 36b of the distal region 36. Thus, the at least one electrode 52 can be supported at the distal tip 37. In one example, the at least one electrode 52 can be disposed in the lumen 39. For instance, the at least one electrode 52 can be supported at the interior surface of the trocar body 31. In other examples, the at least one electrode 52 can be embedded in the trocar body 31. In still other examples, the at least one electrode 52 can be supported at the exterior surface of the trocar body 31. It should be appreciated that the at least one electrode 52 can be oriented along the longitudinal direction L. In other examples, some or all of the at least one electrode 52 can be oriented so as to extend toward the central axis of the trocar 30 as it extends in the distal direction. Alternatively or additionally, some or all of the at least one electrode 52 can be oriented parallel to the central axis of the trocar 30 as it extends in the distal direction.

In some examples, the at least one electrode 52 of the neuro-monitoring system 50 can include a plurality of electrically conductive electrodes 52 circumferentially spaced from each other about the trocar body 31. The electrodes 52 can be spaced equidistantly from each other or variably from each other about or in the trocar body 31 as desired. The number of electrodes 52 and the distance between each of the electrodes can be input and stored and/or otherwise programmed in memory for access by the processor. Each electrode can extend in the trocar body 31, such as in either or both of the first portion 36a and the second portion 36b of the distal region 36. Thus at least a portion up to an entirety of each of the electrodes 52, for instance at the second portion 36b, can taper toward each other as they extend in the distal direction. Further, at least a portion up to an entirety of each of the electrodes 52, for instance at the first portion 36a, can extend parallel to the others of the electrodes as they extend in the distal direction.

A respective neuro-monitoring electrical lead can extend from each electrode 52 to the processor, such that each electrode 52 is in electrical communication with the processor. Alternatively, the electrodes 52 can be monolithic with the leads. Each electrode can comprise a conductive material, such as silver, copper, gold, aluminum, platinum, stainless steel, or the like. If desired, a portion of each electrode can be insulated by a dielectric coating as desired so as to protect the electrically conductive electrode 52. The electrically conductive electrode 52 can define an exposed tip that is not coated by a dielectric. extends out from the dielectric coating.

As the trocar 30 is advanced through the tissue, each electrically conductive electrode 52 can be provided with electrical current. When the distal region 36 of the trocar body 31, and thus each electrode 52, approaches a nerve, the nerve may be stimulated by the electrical current. At a predetermined electrical current, the degree of stimulation to the nerve is related to the distance between the distal tip 37 (and thus each electrically conductive electrode 52) and the nerve. Stimulation of the nerve may be measured by, e.g., visually observing the patient's leg for movement, or by measuring muscle activity through electromyography (EMG) or various other known techniques. Once nerve stimulation is observed or otherwise determined, the distance from the distal region 36 to the nerve can be calculated based on the strength of the electrical current emitted at the at least one electrode 52. This measurement can be referred to as a time of flight mapping method. It should be appreciated that the distance from the distal region 36 to an anatomical structure as used herein can apply to a distance from the respective electrical electrode(s) 52 to the anatomical structure, or a distance from the tip 37 to the anatomical structure based on a known distance from the respective electrical electrode(s) 52 to the tip 37.

The surgical system 25 can alternatively or additionally perform another mapping technique, known as a signal strength mapping method. Under this method, the position of insertion of the trocar 30 into the patient anatomy is known, for instance by monitoring the reference array 40. Thus, the position of the at least one electrode 52 or the distal portion 36 (including the tip 37) is likewise known based on the known positional relationship between the at least one electrode or distal portion from the reference array 40. At the known position, the electrical current emitted by the at least one electrode 52 can be increased until the nerve is stimulated by the electrical current. As described above, stimulation of the nerve may be measured by, e.g., visually observing the patient's leg for movement, or by measuring muscle activity through electromyography (EMG) or various other known techniques. Once nerve stimulation is observed or otherwise determined, the distance of the nerve from the distal region 36 to the nerve can be calculated based on the known position of the electrode and distal portion 36, and the strength of the electrical current that caused the nerve to transition from a non-stimulated state to a stimulated state.

Utilizing the neuro-monitoring system 50 may provide the operator with added guidance for driving the trocar 30 to the desired target anatomical site through Kambin's triangle. With each movement, the operator may be alerted when the tip of the first dilator tube approaches or comes into contact with a nerve. The operator may use this technique alone or in conjunction with other positioning assistance as described herein. The amount of current applied to each electrode 52 may be varied depending on the preferred sensitivity. Naturally, the greater the current supplied, the greater nerve stimulation will result at a given distance from the nerve. In some examples the current applied to each conductive electrode 52 can be a constant current. In other examples the current applied to each conductive electrode 52 can be periodic or irregular. Alternatively, pulses of current may be provided only on demand from the operator.

It is appreciated that a distance can be determined from a given electrically conductive electrode 52 (and thus of the tip 37) to a nerve, and stored in memory. When the distance is greater than a predetermined threshold, the processor can conclude that the actual trajectory of the trocar 30 is different of the desired trajectory of the trocar 30 that extends through Kambin's triangle. The system 25 can therefore provide feedback to the operator to change the actual trajectory of the trocar 30. The feedback can be provided to a feedback device 48 in one example. When the trocar 30 includes a plurality of electrically conductive electrodes 52, the processor can determine the distance from each of the electrodes 52 to the nerve using any technique described above. Further, the position position (including location and orientation) of each of the conductive electrodes 52 is known and can be stored in memory.

Therefore, based on the distance from the electrodes 52 to the nerve, and the known position of each of the electrodes 52 relative to each other, the location of the nerve relative to the trocar 30 can be determined by the processor, for instance by triangulating the distances of each of the electrodes to the nerve based on the known distance of each locations of the trocar 30 relative to each of the electrodes 52. Thus, neuro-monitoring or electrical guidance of the system 25 as achieved by the conductive electrodes 52 and the processor can determine the location of the nerve. The location of the nerve can be displayed to the operator in any manner described herein (see, e.g., FIG. 4B). It should be appreciated that the system 25 can employ the neuro-monitoring electrodes 52, the camera 51, or a combination of both the camera 51 and the neuro-monitoring electrodes 52. Thus, the actual trajectory of the trocar 30 can be guided by the electrical signals from the electrodes 52 and not by the optical signals from the camera 51. Alternatively, the actual trajectory of the trocar 30 can be guided by the optical signals from the camera 51 and not by the electrical signals from the electrodes 52. Alternatively still, the actual trajectory of the trocar 30 can be guided by both the optical signals from the camera 51 and by the electrical signals from the electrodes 52. If desired, the neuro-monitoring leads, and the communication cable 38 can be bundled into a single multicore cable as desired, wherein the multicore cable extends from the camera 51 and electrodes 52 out the trocar to the processor or other component of the computing device as desired.

Regardless of the method used to determine the distance between the trocar 30 and a given nerve, the processor can actuate the vibrating actuator 55 (see FIG. 5A) to vibrate when the trocar 30 is within a predetermined distance of the nerve, thereby alerting the operator to change the trajectory of the trocar 30. The trajectory of the trocar 30 can be changed in real time, or the trocar 30 can be backed out of the anatomy along the proximal direction, and subsequently advanced in the distal direction along a new trajectory.

As described above, the surgical system 25 can include a plurality of sensors that are of a different type from each other, and are configured to sense different parameters. For instance, the different parameters can include any one or more up to all of a position of the trocar, an orientation of the trocar, a property of tissue proximate to the trocar (for instance to identify tissue type), and a distance between a tissue of interest, such as a nerve, to the trocar 30. A processor can be coupled to at least one or more up to all of the sensors to determine the parameter, and/or overlay on the display graphical representations of data from each of the plurality of sensors to provide augmented visualization of the trocar in real-time as it is driven toward and/or through Kambin's triangle. The processor can further overlay a pre-operative image of the anatomy onto the real-time image from the camera of the anatomy so as to provide augmented visualization of the anatomy in the field of view of the camera.

As described above, the surgical system 25 can include at least one feedback device 48 (see FIG. 3A) that is in communication with the processor, and can provide feedback to the operator if it is determined that the actual trajectory of the trocar 30 is different than the desired trajectory or a tolerance value of the desired trajectory. The feedback device 48 can be in communication with the processor, and actuated for instance if it is determined that the actual trajectory of the trocar 30 does not pass through Kambin's triangle. The feedback device 48 can also be actuated if it is determined that the trocar 30 is within a predetermined distance of or is otherwise approaching the exiting nerve 21 or the traversing nerve root 23. The feedback device 48 can be disposed anywhere as desired. For instance, the feedback device 48 can be carried by the trocar handle 32 in one embodiment. In one example, the feedback device 48 can be a haptic feedback device, such as the vibrating actuator 55 (see FIG. 5A) that vibrates when actuated. Alternatively or additionally still, the feedback device 48 can emit an audible signal. The audible signal can include correction instructions to change the actual trajectory to the desired trajectory.

It should be appreciated that both the camera 51 and the neuro-monitoring system 50 can provide input to the processor of the real-time position of the anatomical structure of and surrounding Kambin's triangle including the structure pre-operatively imaged and shown in FIG. 4A. The image of FIG. 4A can be obtained using any suitable pre-operative imaging techniques of the anatomy, for instance using computed tomography (CT), X-Ray, magnetic resonance imaging (MRI), or the like. As will now be described with reference to FIG. 4B, the anatomical structure of FIG. 4A can be overlaid on the camera image as the trocar 30 is driven toward and through Kambin's triangle. Further, to assist with guidance of the trocar, the anatomical structure can be labeled on the display 46 as the trocar 30 is driven toward and through Kambin's triangle. Thus, the user can identify the anatomical structure in the field of view of the camera, and adjust the trajectory of the trocar 30 as desired. Alternatively or additionally, the location of the nerves 21 and 23 as determined using the neuro-monitoring system can be overlaid on the camera image.

Thus, referring now to FIG. 4B, the feedback device 48 can be in the form of a surgical navigation display 46 of images overlaid on the real-time camera image 49 from the camera 51. Further, the anatomical structures provided on the display as graphical representations of data from one or more up to all of the camera 51 (FIG. 3A), the reference array 40 (FIG. 3A), and the neuro-monitoring system 50 can include highlighted regions of an image captured by the camera that denotes the presence of any of a type of tissue (e.g., muscle, bone, and nerve) and an anatomical structure (e.g., any one or more of the vertebrae, exiting nerve, and nerve root). The display 46 can include anatomical structure from pre-operative images of the anatomy (see, e.g., FIG. 4A), for instance using computed tomography (CT), X-Ray, magnetic resonance imaging (MRI), or the like. Further, the processor can receive the real-time images of the anatomical structure from the camera 51 as the trocar 30 is driven toward Kambin's triangle. As described herein, the processor can update the display of any one or more up to all of the position and size and shape of the anatomical structure in FIG. 4A based on the real-time data from the images of the camera 51 of the anatomical structure as the trocar 30 travels toward Kambin's triangle, and update the display on the display 46 of FIG. 4B.

Further, as will now be described with continuing reference to FIG. 4B, the processor can display on the camera image 49 a graphical indication of a state of alignment between either or both of first and second locations of the trocar 30 with respect to desired locations of either or both of the first and second locations of the trocar 30. As described above with reference to FIG. 3A, the reference array 40 can provide the orientation of the trocar 30. In one example the first location can be spaced from the second location in the distal direction. For instance, the first location can be defined by the tip 27 of the trocar 30, and the second location can be defined by the trocar handle 32. In some examples, the first and second locations of the trocar 30 can be disposed along the central axis 45 of the trocar.

For at least one or both of the first and second locations of the trocar 30, the display 46 can include visual indicia such as one or more up to all of 1) the location of the trocar 30, 2) the position of the location of the trocar 30 relative to the anatomical structure, and 3) whether the location of the trocar is substantially aligned with a desired position of the location of the trocar 30. The indicia can further distinguish the first location from the trocar and the second location of the trocar 30. In one example, a first image icon 62 can identify the first location of the trocar, and a second image icon 64 different than the first image characteristic can identify the second location of the trocar. For instance, the first and second image icons 62 and 64 can be one or more of a size and shape. In one example, the one of the first and second image icons (such as the first image icon 62) can be configured as a circle, and the other of the first and second image icons (such as the second image icon 64) can be configured as a crosshair. Alternatively, the first and second image icons can be configured as different colors, different line thicknesses, or the like.

As the trocar 30 is driven through the anatomy toward and through Kambin's triangle, the first and second image icons have a relative position on the display that indicates whether the first location is aligned with the second location along the actual trajectory of the trocar 30 as the trocar 30 is driven into the anatomy. For instance, the first image icon is spaced from the second image icon on the display 46 when the first location is out of alignment with the second location with respect to the actual trajectory of the trocar 30. Further, the distance direction along which the first image icon is spaced from the position of the second image on the display 46 informs the operator of a correctional direction and distance of either or both of the first and second locations in order to achieve alignment of the first and second ends along the actual trajectory. Moving the first location of the trocar 30 will cause the first image icon 62 to correspondingly to move on the display 46. Similarly, moving the second location of the trocar 30 will cause the second image icon 64 to correspondingly to move on the display 46. The processor can further actuate the vibrating actuator 55 (see FIG. 5A) to vibrate when the first location of the trocar 30 is out of alignment with the second location of the trocar along the actual trajectory of the trocar 30.

The first and second image icons can merge when the first and second locations are aligned with each other along the actual trajectory. For instance, the crosshair can be centered inside the circle when the first and second locations are aligned with each other along the actual trajectory. When the first and second locations of the trocar 30 are aligned with each other, the actual trajectory of the trocar 30 is along the central axis 45 of the trocar. Further, when the first and second locations of the trocar 30 are aligned with each other, the center of the display from the camera 51 can be disposed on the trajectory of travel of the trocar 30 when the camera 51 is centered on the central axis of the trocar and faces the distal direction. Thus, the operator can visually inspect the display 46 to assess whether the actual trajectory is aligned with Kambin's triangle, or if the actual trajectory is aligned with an anatomical structure other than Kambin's triangle, such as a nerve 66 (for example the exiting nerve 21 or the traversing nerve root 23), bony tissue 68, and soft tissue 70. The operator can then adjust the actual trajectory to a desired trajectory through Kambin's triangle. As is described below, the surgical system 25 can compensate for situations where the camera 51 is positioned offset from the central axis of the trocar and/or angulated with respect to the distal direction, so that the display 46 shows the image from the camera 51 as if the camera were positioned on the central axis and facing the distal direction. Additionally, either or both of the first and second image icons 62 and 64 can be a predetermined color when the actual trajectory is at least substantially aligned with the desired trajectory. “Substantially” in this context can mean that the trocar 30 is within a tolerance of the predetermined desired trajectory through Kambin's triangle so that the trocar 30 will not contact either of the nerves 21 and 23 or other tissue with which it is desirable to avoid contact with the trocar 30. For instance, either or both of the first and second image icons 62 and 64 can be a first color, such as red, when the actual trajectory is not substantially aligned with the predetermined desired trajectory. Either or both of the first and second image icons 62 and 64 can be a second color, such as green, when the actual trajectory is substantially aligned with the predetermined desired trajectory. When the first and second locations are aligned along the actual trajectory, and the actual trajectory is substantially aligned with the desired trajectory, then the actual trajectory of the trocar 30 is through Kambin's triangle.

Referring now to FIGS. 5A-7 generally, the surgical system 25 can further include a flexible surgical access port 130 that is configured to provide an access path to the spine through Kambin's triangle 24. The flexible surgical access port 130 can include a collar 128 and a flexible surgical access body 136 that extends generally distally from the collar 128. The collar 128 can be configured as an annulus that defines a bore 132 open to the flexible body 136 The bore 132 can be cylindrical or alternatively shaped as desired. Further, the collar 128 can be rigid or flexible. The flexible body 136 extends from the collar 128 along a central axis 134. The flexible surgical access port 130 can define a central axis 134 that extends through the flexible body 136. The flexible body 136 can define a proximal end 138a and a distal end 138b that is opposite the proximal end 138a along the central axis 134. A lumen 140 can extend through the flexible surgical access port 130 from its proximal end to its distal end. Thus, the lumen 140 can also extend through the entire length of the flexible body 136 from the proximal end 138a to the distal end 138b along the central axis 134.

The surgical access port 130 can be coupled to the trocar 30 prior to driving the trocar 30 into the patient's anatomy and to/through Kambin's triangle. Thus, an access assembly 43 that includes the trocar 30 and the surgical access port 130 can be driven to and through Kambin's triangle along the desired trajectory in the manner described herein. In particular, the trocar 30 is driven to/through Kambin's triangle, and the surgical access port 130 travels with the trocar 30. In one example, the trocar 30 can be inserted through the surgical access port 130 so as to couple the surgical access port 130 to the trocar 30. In particular, the distal tip 37, along with the distal region 36 and the trocar shaft 34, can be driven distally through the lumen 140 until the distal tip 37 extends out from the distal end 138b of the flexible body 36. In examples whereby the flexible body 36 extends from the port handle 152 and port grommet 154, the distal end 36 and the trocar shaft 34 can be driven distally through the port handle 152 and the port grommet 154 and then through the lumen 140 until the port handle 152 abuts or nests with the trocar handle 32. In one example, the trocar 30 can be releasably locked to the surgical access port 130 as the assembly 43 is driven to and through Kambin's triangle.

It should be appreciated that the access assembly 43 including the trocar 30 and the surgical access port 130 can be the first instruments inserted into the patient during the surgical procedure. Once the trocar 30 has been driven past Kambin's triangle to the intervertebral disc space, the trocar 30 can be unlocked from the surgical access port 130 and can subsequently be removed from the surgical access port 130, and the surgical implements to perform the surgical procedure in the disc space can be delivered through the lumen 140 of the surgical access port 130.

The proximal end 138a can be coupled to the collar 128 in any manner as desired, such that the lumen 140 is in communication with the bore 132 of the collar 128. In particular, a central axis of the bore 132 can be aligned with the central axis 134 of the flexible surgical access port 130. The surgical system 25 can include a handle 142 that is configured to support the flexible surgical access port 130. In one example, the handle 142 can be coupled to the collar 128 in any suitable manner so as to direct the flexible surgical access port 130 toward a target anatomical site such as Kambin's triangle 24. Thus, an apparatus such as a surgical instrument or implant can be inserted distally through the bore 132 and into the lumen 140 toward the spine. The collar 128 can define a proximal end of the flexible surgical access port 130.

Referring now to FIGS. 6A-6B in particular, the flexible body 136, and thus the flexible surgical access port 130, can advantageously be configured to expand radially from a first configuration having first cross-sectional dimension to a second or expanded configuration having a second cross-sectional dimension that is greater than the first cross-sectional dimension. The first and second cross-sectional dimensions are measured along the same direction and can extend through the central axis 134. In some examples, the first and second cross-sectional dimensions can be configured as diameters when the flexible body 136 is circular in cross-section. The flexible body 136 can define any suitable shape as desired. The flexible body 136, and thus the flexible surgical access port 130, can be woven or nonwoven as desired. When woven, the flexible body 136 can be made from any suitable pattern of woven fibers 144 that define a weave pattern. Description of the flexible body 136 herein can apply with equal force and effect to the flexible surgical access port 130. In one example, the fibers 144 can be interwoven so as to define a mesh. In other examples, the fibers 144 can define a lattice. Thus, the fibers 144 can intersect at respective angles of intersection that can change as the flexible body 136 expands radially. Thus, one or more of the angles of intersection can be measured to determine a quantification of an outer diameter of the flexible body 136. In still other examples, the fibers 144 can be braided. For instance, the fibers 144 can be helically wound to define a braid.

During operation, the flexible body 136 can be collapsed in the first configuration, and can be urged to a normal relaxed geometric configuration. In the normal relaxed geometric configuration, the flexible body 136 is no longer collapsed, but has not been expanded beyond its normal relaxed geometric shape. For instance, when the flexible body 136 is configured as a cylindrical body, the flexible fibers 144 can be collapsed in the first configuration and thus not define a cylinder. The flexible body 136 can be urged to its normal and relaxed cylindrical geometric shape if desired. However, the flexible body 136 has not yet expanded. Thus, the first configuration can either be collapsed or in its normal relaxed geometric shape in the first configuration. The flexible body 136 is configured to expand beyond the first configuration to an expanded position whereby at least a portion of the flexible body is expanded beyond the normal relaxed geometric configuration. Expansion of the flexible body 136 to the second position can be along a direction that is perpendicular to the central axis 134.

The surgical system 25 can include surgical equipment 146 that is configured to be driven distally through the lumen 140. The surgical equipment can have a cross-sectional dimension that is greater than the cross-sectional dimension of the flexible body when the flexible body is in the first configuration. The cross-sectional dimension of the surgical equipment 146 is oriented in the same direction as the cross-sectional dimension of the flexible body 136. Thus, the surgical equipment 146 apply a radially outward force that urges the flexible body 136 to expand to a second configuration that is beyond its normal relaxed geometric configuration. When the flexible body 136 defines a lattice structure, the surgical equipment can urge the flexible body 136 to vary the angles of intersection so as to expand the flexible body 136 to the second configuration. Thus, in some examples the flexible body 136 can expand to the second configuration without substantial expansion of the fibers 144 along their respective lengths. In this regard, the fibers 144 can be substantially rigid along their lengths.

In other examples the fibers 144 can be expandable along their lengths so as to expand flexible body 136. For instance, the fibers 144 can extend circumferentially about the central axis, such that expansion of the fibers 144 along their lengths causes the flexible body 136 to expand radially. For instance, the fibers 144 can be defined by an elastically deformable elastomer that can define a braid, a mesh, a lattice structure, or any suitable alternative woven structure as desired. Thus, elongation of the fibers 144 can contribute to the movement of the flexible body 136 from the first configuration to the second configuration. In other examples, the flexible body 136 can be nonwoven and made from an expandable material. The flexible body 136 can be elastic so as to move toward or to the first configuration after being expanded to the second configuration. In other examples, the flexible body 136 can be substantially inelastic such that compressive forces from surrounding anatomical tissue can cause the flexible body 136 to move toward or to the first configuration from the second configuration. The fibers can be made of Nickel-Titanium (NiTi) or any suitable alternative material as desired. In one example, the filaments can have a shape memory, such that as the flexible body is deflected into a desired shape, the flexible body remains in the desired shape.

The term “substantially,” “approximately,” and derivatives thereof, and words of similar import, when used to described sizes, shapes, spatial relationships, distances, directions, expansion, and other similar parameters includes the stated parameter in addition to a range up to 10% more and up to 10% less than the stated parameter, including up to 5% more and up to 5% less, including up to 3% more and up to 3% less, including up to 1% more and up to 1% less.

With continuing reference to FIGS. 4A-4C, the surgical equipment 146 can be sized to be inserted through the bore 132 of the collar 128 (see FIG. 3). Further, when the flexible body 136 is in the first configuration, the first cross-sectional dimension is less than the cross-sectional dimension of the bore 132 of the collar 128. Thus, when the surgical equipment 146 is driven through the bore 132 and into the lumen 140, the flexible body 136 expands to the second configuration whereby the second cross-sectional dimension is no greater than that of the bore 132 in some examples. In other examples, it is recognized that the surgical equipment 146 can be inserted through the bore 132 in a first orientation, and subsequently iterated to a second orientation in the lumen 140 that causes the lumen 140 to expand to the second cross-sectional dimension that is greater than the cross-sectional dimension of the bore 132. The flexible body 136 can abut at least a portion up to an entirety of the surgical equipment that caused the flexible body 136 to expand.

Accordingly, during operation, the surgical equipment 146 such as a surgical instrument or implant can be driven through the bore 132 and into the flexible body 136. The surgical equipment 146 can be sized to fit through the bore 132, and sized greater than the first cross-sectional dimension of the flexible body 136. Thus, as the surgical equipment 146 is driven through the lumen 140, a force from the surgical equipment 146 urges a local region of the flexible body 136 to expand radially from the first configuration to the second configuration. The local region can include an aligned location of the flexible body 136 that is aligned with the surgical equipment 146 and regions adjacent the aligned location that are urged to expand by the force from the surgical equipment 146 as it travels through the lumen 140. That is, the region of the flexible body 136 that are aligned with the surgical equipment 146 or adjacent the portion of the flexible body 136 that is aligned with the surgical equipment 146 can expand outward in order to enlarge the lumen 140 to accommodate the surgical equipment whose cross-sectional dimension is greater than that of the flexible body 136 when the flexible body is in the first configuration. Typically, the aligned region will expand a greater amount than the adjacent region. Once the force from the surgical equipment 146 is removed, for instance when the surgical equipment has travelled to a location remote from the aligned region, the locations of the flexible body 136 that have expanded can return toward or to the first configuration. Remote regions of the flexible body 136 that are remote from the surgical equipment 146 can be in the first configuration.

Thus, as the surgical equipment 146 is driven through the lumen 140, previously expanded regions of the surgical equipment 146 can either remain in the second configuration or return from the second configuration toward or to the first configuration as the surgical equipment 146 travels distally along the lumen 140 a sufficient distance such that portions of the flexible body 136 that previously defined local regions now define remote regions, whereby the surgical equipment 146 no longer exerts a force on the remote regions sufficient to cause the remote regions to expand from the first configuration. The local regions 136 of the flexible body move distally as the surgical equipment is advanced distally in the lumen 140. Conversely, the local regions 136 of the flexible body 136 move proximally as the surgical equipment is advanced proximally in the lumen 140. As the surgical equipment 146 travels in the lumen 140, locations of the flexible body 136 that were urged by the surgical equipment 146 to expand to the second configuration can return toward or to the first configuration when the surgical equipment 146 has travelled to a position remote of the locations such that the locations define remote regions. Natural biasing forces of the flexible body 136 can urge the flexible body 136 toward or to the first configuration after the surgical equipment 146 has passed by. Therefore, the surgical equipment 146 urges the flexible body 136 to expand as the surgical equipment 146 travels distally and proximally, selectively, in the lumen 140. It should be appreciated that the local expansion of the flexible body 136 can thus be momentary, as the local regions become remote regions that then return toward or to the first configuration once the surgical equipment has passed by.

As a result, anatomical tissue surrounding the flexible body 136 undergoes only momentary compression due to the momentary expansion of the flexible body 136 from the first configuration to the second configuration. At some regions surrounding the flexible body 136, the surrounding anatomical tissue can include fatty tissue and musculature of the patient. At other regions of the flexible body 136, the surrounding tissue can include a nerve such as either or both of the exiting nerve 21 and the traversing nerve root 23 that partially define Kambin's triangle. Advantageously, large surgical equipment 146 can pass through the flexible body 136 while causing only momentary contact between the flexible body 136 and the nerve. A rigid conduit, on the contrary, that is sized to receive the large surgical equipment would bear against the nerve for as long as the conduit were in place during the surgical procedure. Thus, the surgical system 25 prevents the nerve from undergoing prolonged compression during spinal surgery.

Referring now to FIG. 6C, the flexible body 136 can also be configured to deflect along a direction perpendicular to the central axis 134. Accordingly, when the surgical equipment 146 has a curvature and is inserted into the lumen 140, the surgical equipment 146 can correspondingly impart a curvature to the flexible body 136. Thus, the central axis 134 can extend along one or more curved paths. When the surgical equipment 146 is removed from the lumen 140, the flexible body 136 can return toward or to the first configuration. Alternatively or additionally, a surgical equipment 146 inserted into the lumen 140 along a direction that is angularly offset with respect to the central axis 134 in a select direction can cause either or both of at least a portion of the central axis 134 and the distal end of the flexible body 136 to correspondingly deflect in the select direction. Therefore, the surgical equipment 146 can change a trajectory of the lumen that is defined by a direction that separates the proximal end 138a to the distal end 138b from a first trajectory to a second trajectory. The first trajectory can be the trajectory through Kambin's triangle as defined by the trocar 30. Moving from the first trajectory to the second trajectory can be advantageous when it is desired to perform one or more procedures on different areas of the spine. The flexible body 136 can define the first trajectory when it is in the first configuration. The distal end 138b of the flexible body 136 can also define the distal end of the flexible surgical access port 130.

Referring now to FIG. 5A, and as described above, the surgical equipment 146 of the surgical system 25 can include the trocar 30 or any suitable alternative access member that can be configured to establish a trajectory to a target anatomical site. In some examples, the trocar 30 can extend through the lumen 140 while the flexible body 136 remains in the first configuration. In other examples, the trocar 30 can cause the flexible body 136 to expand beyond the first configuration to the second configuration. While the trocar 30 extends through the lumen 140, the trocar 30 can be driven through the anatomy of the patient toward a target anatomical site, such as through Kambin's triangle along a desired trajectory in the manner described above. The trocar shaft 34 can be sized to extend through the bore 132 of the collar 128 of the flexible surgical access port 130 (see FIG. 5B), which can be defined by either or both of a port handle 152 and a proximal port grommet 154 of the flexible surgical access port 130. The flexible body 136 can extend in the distal direction from the proximal port grommet 154. The proximal port grommet 154 can have an inner cross-sectional dimension such as a diameter that is equal to the expanded cross-sectional dimension of the flexible body 136. The flexible surgical access port 130 can further include a distal port grommet that extends distally from the flexible body 136 and has an inner cross-sectional dimension equal to that of the proximal port grommet 154. The distal region 36 of the trocar body 31 and the trocar shaft 34 can be driven distally through the port handle 152 and the proximal port grommet 154 and through the lumen 140 so that the tapered distal portion 136b of the trocar shaft 34 extends distally past the distal end of the flexible body 136. In one example, the target anatomical site is defined by a target surgical location, which in turn can be defined by the superior articular process. Alternatively or additionally, the target surgical location can be defined by the disc space. The trocar handle 32 can seat against or removably interlock with the port handle 152 when the trocar shaft 34 has been fully driven through the flexible body 136. The distal tip 37 of the trocar 30 can extend distal of the access port when the trocar 30 has been fully inserted in the surgical access port 130.

The trocar 30 can be decoupled and removed from the surgical access port by moving the trocar 30 in the proximal direction with respect to the surgical access port until the trocar has been removed 30. The lumen of the surgical access port 130 can then provide a working channel to the intervertebral disc space. The surgical access port 130 can be docked to either or both of the vertebral bodies that define the intervertebral space either before or after the trocar 30 has been decoupled from the surgical access port 130 and removed from the surgical access port 130, The surgical access port 130 can include any suitable docking structure as desired that can releasably secure to the vertebra or vertebrae.

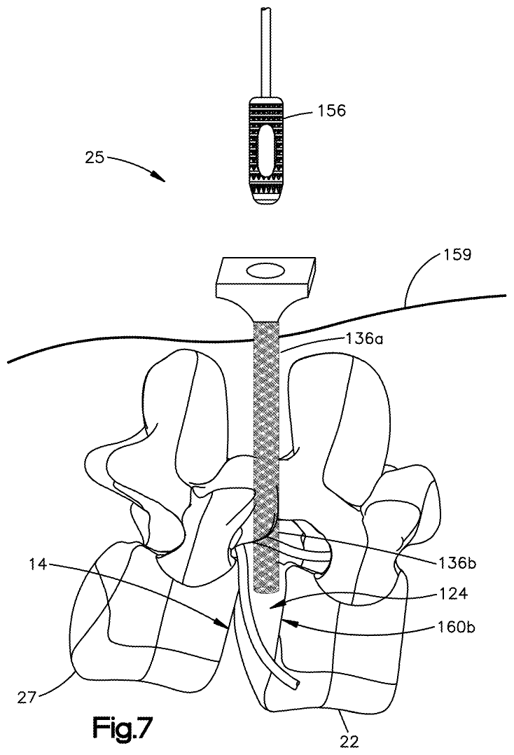

In other examples (see FIG. 7), the surgical system can include first and second surgical access ports 136a and 136b. The trocar 30 can guide the first surgical access port 136a to Kambin's triangle, and can be removed from the first surgical access port 136a, such that the lumen of the first surgical access port 136a provides a working channel to Kambin's triangle. After surgical steps have been performed through the working channel of the first surgical access port 136a, a second surgical access port 136b can be inserted distally through the lumen of the first surgical access port 136a through Kambin's triangle to the intervertebral disc space. The lumen of the second surgical access port 136b thereby defines a working channel to the disc space.

Referring to FIG. 7, in some examples the surgical equipment of the surgical system 25 that can be driven through the lumen of the surgical access port 130 to the intervertebral space can include an intervertebral implant 156 that is sized to be driven distally through the lumen 140. The intervertebral implant 156 can be configured as a spinal fusion cage. Thus, the flexible body 136 can be configured to receive the intervertebral implant 156. The intervertebral implant 156 can travel through the lumen 140 and into the intervertebral disc space. The intervertebral implant 156 can cause the flexible body 136 to expand from the first configuration to the second configuration as the intervertebral implant 156 travels distally through the lumen 140. The implant 156 causes the flexible body 136 to expand 140 at regions adjacent the implant 156 as the implant 156 travels distally through the lumen 140. Regions of the flexible body 136 can return toward or to the first configuration after the intervertebral implant 156 has passed by distally. Thus, any nerves that are compressed due to expansion of the flexible body 136 are only compressed momentarily until the implant 156 has passed by distally.

It should be appreciated that the various surgical equipment of the surgical system 25 can cause the flexible body 136 to expand radially different amounts from the first configuration, and all such degrees of expansion can define the second configuration. The maximum second cross-sectional dimension can be approximately four times the first cross-sectional dimension. By way of example, the flexible body 136 can define a first cross-sectional dimension of approximately 4 mm when in the first configuration, and can define a maximum second cross-sectional dimension of approximately 15 mm when expanded. The surgical access port 130 and first and second surgical access ports are described in U.S. patent application Ser. No. 17/510,709 filed Oct. 26, 2021, the disclosure of which is hereby incorporated by reference as if set forth in its entirety herein.

While the illustrated embodiments and accompanying description make particular reference to application in a spinal surgery procedure, and, in particular, to minimally invasive spinal surgery, the devices, systems, and methods described herein are not limited to these applications.

In some embodiments, intra-operative feedback may be received from at least one surgical instrument regarding positioning of the identified neurological structures; and the patient-specific surgical access plan may be updated.

In some embodiments, real-time positioning of at least one of the identified neurological structures, a surgical instrument, and a patient position may be displayed.

Referring now to FIGS. 8A-10 generally, the terms artificial neural network (ANN) and neural network may be used interchangeably herein. An artificial neural network may be configured to determine a classification (e.g., type of object) based on input image(s) or other sensed information. An artificial neural network is a network or circuit of artificial neurons or nodes, and it may be used for predictive modeling.

The prediction models may be and/or include one or more neural networks (e.g., deep neural networks, artificial neural networks, or other neural networks), other machine learning models, or other prediction models.

Disclosed implementations of artificial neural networks may apply a weight and transform the input data by applying a function, this transformation being a neural layer. The function may be linear or, more preferably, a nonlinear activation function, such as a logistic sigmoid, Tanh, or a rectified linear unit (ReLU) function. Intermediate outputs of one layer may be used as the input into a next layer. The neural network through repeated transformations learns multiple layers that may be combined into a final layer that makes predictions. This learning (i.e., training) may be performed by varying weights or parameters to minimize the difference between the predictions and expected values. In some embodiments, information may be fed forward from one layer to the next. In these or other embodiments, the neural network may have memory or feedback loops that form, e.g., a neural network. Some embodiments may cause parameters to be adjusted, e.g., via back-propagation.

An artificial neural network is characterized by features of its model, the features including an activation function, a loss or cost function, a learning algorithm, an optimization algorithm, and so forth. The structure of an artificial neural network may be determined by a number of factors, including the number of hidden layers, the number of hidden nodes included in each hidden layer, input feature vectors, target feature vectors, and so forth. Hyperparameters may include various parameters which need to be initially set for learning, much like the initial values of model parameters. The model parameters may include various parameters sought to be determined through learning. And the hyperparameters are set before learning, and model parameters can be set through learning to specify the architecture of the artificial neural network.

Learning rate and accuracy of an artificial neural network rely not only on the structure and learning optimization algorithms of the artificial neural network but also on the hyperparameters thereof. Therefore, in order to obtain a good learning model, it is important to choose a proper structure and learning algorithms for the artificial neural network, but also to choose proper hyperparameters.

The hyperparameters may include initial values of weights and biases between nodes, mini-batch size, iteration number, learning rate, and so forth. Furthermore, the model parameters may include a weight between nodes, a bias between nodes, and so forth.

In general, the artificial neural network is first trained by experimentally setting hyperparameters to various values, and based on the results of training, the hyperparameters can be set to optimal values that provide a stable learning rate and accuracy.

Some embodiments of models 264 in system 25 depicted in FIG. 8B may comprise a convolutional neural network (CNN). A convolutional neural network may comprise an input and an output layer, as well as multiple hidden layers. The hidden layers of a convolutional neural network typically comprise a series of convolutional layers that convolve with a multiplication or other dot product. The activation function is commonly a ReLU layer, and is subsequently followed by additional convolutions such as pooling layers, fully connected layers and normalization layers, referred to as hidden layers because their inputs and outputs are masked by the activation function and final convolution.

The convolutional neural network computes an output value by applying a specific function to the input values coming from the receptive field in the previous layer. The function that is applied to the input values is determined by a vector of weights and a bias (typically real numbers). Learning, in a neural network, progresses by making iterative adjustments to these biases and weights. The vector of weights and the bias are called filters and represent particular features of the input (e.g., a particular shape).

In some embodiments, the learning of models 264 may be of reinforcement, supervised, semi-supervised, and/or unsupervised type. For example, there may be a model for certain predictions that is learned with one of these types but another model for other predictions may be learned with another of these types.

Supervised learning is the machine learning task of learning a function that maps an input to an output based on example input-output pairs. It may infer a function from labeled training data comprising a set of training examples. In supervised learning, each example is a pair consisting of an input object (typically a vector) and a desired output value (the supervisory signal). A supervised learning algorithm analyzes the training data and produces an inferred function, which can be used for mapping new examples. And the algorithm may correctly determine the class labels for unseen instances.

Unsupervised learning is a type of machine learning that looks for previously undetected patterns in a dataset with no pre-existing labels. In contrast to supervised learning that usually makes use of human-labeled data, unsupervised learning does not via principal component (e.g., to preprocess and reduce the dimensionality of high-dimensional datasets while preserving the original structure and relationships inherent to the original dataset) and cluster analysis (e.g., which identifies commonalities in the data and reacts based on the presence or absence of such commonalities in each new piece of data).

Semi-supervised learning makes use of supervised and unsupervised techniques.

Models 264 may analyze made predictions against a reference set of data called the validation set. In some use cases, the reference outputs resulting from the assessment of made predictions against a validation set may be provided as an input to the prediction models, which the prediction model may utilize to determine whether its predictions are accurate, to determine the level of accuracy or completeness with respect to the validation set data, or to make other determinations. Such determinations may be utilized by the prediction models to improve the accuracy or completeness of their predictions. In another use case, accuracy or completeness indications with respect to the prediction models' predictions may be provided to the prediction model, which, in turn, may utilize the accuracy or completeness indications to improve the accuracy or completeness of its predictions with respect to input data. For example, a labeled training dataset may enable model improvement. That is, the training model may use a validation set of data to iterate over model parameters until the point where it arrives at a final set of parameters/weights to use in the model.

In some embodiments, training component 232 depicted in FIG. 8B may implement an algorithm for building and training one or more deep neural networks. A used model may follow this algorithm and already be trained on data. In some embodiments, training component 232 may train a deep learning model on training data 262 providing even more accuracy, after successful tests with these or other algorithms are performed and after the model is provided a large enough dataset.

A model implementing a neural network may be trained using training data of storage/database 262. The training data may include many anatomical attributes. For example, this training data obtained from prediction database 260 may comprise hundreds, thousands, or even many millions of pieces of information (e.g., images, scans, or other sensed data) describing portions of a cadaver or live body, to provide sufficient representation of a population or other grouping of patients. The dataset may be split between training, validation, and test sets in any suitable fashion. For example, some embodiments may use about 60% or 80% of the images or scans for training or validation, and the other about 40% or 20% may be used for validation or testing. In another example, training component 232 may randomly split the labelled images, the exact ratio of training versus test data varying throughout. When a satisfactory model is found, training component 232 may train it on 95% of the training data and validate it further on the remaining 5%.

The validation set may be a subset of the training data, which is kept hidden from the model to test accuracy of the model. The test set may be a dataset, which is new to the model to test accuracy of the model. The training dataset used to train prediction models 64 may leverage, via training component 232, an SQL server and a Pivotal Greenplum database for data storage and extraction purposes.

In some embodiments, training component 232 may be configured to obtain training data from any suitable source, e.g., via prediction database 260, electronic storage 222, external resources 224 (e.g., which may include sensors, scanners, or another device), network 270, and/or user interface device(s) 218. The training data may comprise captured images, smells, light/colors, shape sizes, noises or other sounds, and/or other discrete instances of sensed information.

In some embodiments, training component 232 may enable one or more prediction models to be trained. The training of the neural networks may be performed via several iterations. For each training iteration, a classification prediction (e.g., output of a layer) of the neural network(s) may be determined and compared to the corresponding, known classification. For example, sensed data known to capture a closed environment comprising dynamic and/or static objects may be input, during the training or validation, into the neural network to determine whether the prediction model may properly predict a path for the user to reach or avoid said objects. As such, the neural network is configured to receive at least a portion of the training data as an input feature space. Once trained, the model(s) may be stored in database/storage 264 of prediction database 260, as shown in FIG. 8B, and then used to classify samples of images or scans based on visible attributes.

Electronic storage 222 of FIG. 8B comprises electronic storage media that electronically stores information. The electronic storage media of electronic storage 222 may comprise system storage that is provided integrally (i.e., substantially non-removable) with system 25 and/or removable storage that is removably connectable to system 25 via, for example, a port (e.g., a USB port, a firewire port, etc.) or a drive (e.g., a disk drive, etc.). Electronic storage 222 may be (in whole or in part) a separate component within system 25, or electronic storage 222 may be provided (in whole or in part) integrally with one or more other components of system 25, such as a user interface device 218, processor 221, and the like. As shown in FIG. 8A, the processor 221 can further receive images from pre-operative imaging devices 223, such as a CT scan, MM, X-Ray, or the like. The processor 221 can further receive data from either or both of the camera and neuro-monitoring electrodes of the trocar 30, and identify the first and second locations with the first and second icons 62 and 64. The processor 221 can further provide real-time surgical navigation 227, for instance on the display 46 (see FIG. 4B).

Referring again to FIG. 8B, in some embodiments the electronic storage 222 may be located in a server together with processor 221, in a server that is part of external resources 224, in user interface devices 218, and/or in other locations. Electronic storage 222 may comprise a memory controller and one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 222 may store software algorithms, information obtained and/or determined by processor 221, information received via user interface devices 218 and/or other external computing systems, information received from external resources 224, and/or other information that enables system 25 to function as described herein.

External resources 224 may include sources of information (e.g., databases, websites, etc.), external entities participating with system 25, one or more servers outside of system 25, a network, electronic storage, equipment related to Wi-Fi technology, equipment related to Bluetooth® technology, data entry devices, a power supply (e.g., battery powered or line-power connected, such as directly to 110 volts AC or indirectly via AC/DC conversion), a transmit/receive element (e.g., an antenna configured to transmit and/or receive wireless signals), a network interface controller (NIC), a display controller, a graphics processing unit (GPU), and/or other resources. In some implementations, some or all of the functionality attributed herein to external resources 224 may be provided by other components or resources included in system 25. Processor 221, external resources 224, user interface device 218, electronic storage 222, a network, and/or other components of system 25 may be configured to communicate with each other via wired and/or wireless connections, such as a network (e.g., a local area network (LAN), the Internet, a wide area network (WAN), a radio access network (RAN), a public switched telephone network (PSTN), etc.), cellular technology (e.g., GSM, UMTS, LTE, 5G, etc.), Wi-Fi technology, another wireless communications link (e.g., radio frequency (RF), microwave, infrared (IR), ultraviolet (UV), visible light, cm wave, mm wave, etc.), a base station, and/or other resources.

The user interface device(s) 218 of system 25 may be configured to provide an interface between one or more users and system 25 user interface devices 218 are configured to provide information to and/or receive information from the one or more user interface devices 218 include a user interface and/or other components. The user interface may be and/or include a graphical user interface configured to present views and/or fields configured to receive entry and/or selection with respect to particular functionality of system 25, and/or provide and/or receive other information. In some embodiments, the user interface of user interface devices 218 may include a plurality of separate interfaces associated with processors 221 and/or other components of system 25. Examples of interface devices suitable for inclusion in user interface device 218 include a touch screen, a keypad, touch sensitive and/or physical buttons, switches, a keyboard, knobs, levers, a display, speakers, a microphone, an indicator light, an audible alarm, a printer, and/or other interface devices. The present disclosure also contemplates that user interface devices 218 include a removable storage interface. In this example, information may be loaded into user interface devices 218 from removable storage (e.g., a smart card, a flash drive, a removable disk) that enables users to customize the implementation of user interface devices 218.

In some embodiments, user interface devices 218 are configured to provide a user interface, processing capabilities, databases, and/or electronic storage to system 25. As such, user interface devices 218 may include processors 221, electronic storage 222, external resources 224, and/or other components of system 25. In some embodiments, user interface devices 18 are connected to a network (e.g., the Internet). In some embodiments, user interface devices 18 do not include processor 221, electronic storage 222, external resources 224, and/or other components of system 25, but instead communicate with these components via dedicated lines, a bus, a switch, network, or other communication means. The communication may be wireless or wired. In some embodiments, user interface devices 218 are laptops, desktop computers, smartphones, tablet computers, and/or other user interface devices.

Data and content may be exchanged between the various components of the system 25 through a communication interface and communication paths using any one of a number of communications protocols. In one example, data may be exchanged employing a protocol used for communicating data across a packet-switched internetwork using, for example, the Internet Protocol Suite, also referred to as TCP/IP. The data and content may be delivered using datagrams (or packets) from the source host to the destination host solely based on their addresses. For this purpose the Internet Protocol (IP) defines addressing methods and structures for datagram encapsulation. Of course other protocols also may be used. Examples of an Internet protocol include Internet Protocol version 4 (IPv4) and Internet Protocol version 6 (IPv6).

In some embodiments, processor(s) 221 may form part (e.g., in a same or separate housing) of a user device, a consumer electronics device, a mobile phone, a smartphone, a personal data assistant, a digital tablet/pad computer, a wearable device (e.g., watch), AR goggles, VR goggles, a reflective display, a personal computer, a laptop computer, a notebook computer, a work station, a server, a high performance computer (HPC), a vehicle (e.g., embedded computer, such as in a dashboard or in front of a seated occupant of a car or plane), a game or entertainment system, a set-top-box, a monitor, a television (TV), a panel, a space craft, or any other device. In some embodiments, processor 221 is configured to provide information processing capabilities in system 25. Processor 221 may comprise one or more of a digital processor, an analog processor, a digital circuit designed to process information, an analog circuit designed to process information, a state machine, and/or other mechanisms for electronically processing information. Although processor 221 is shown in FIGS. 8A-8B as a single entity, this is for illustrative purposes only. In some embodiments, processor 21 may comprise a plurality of processing units. These processing units may be physically located within the same device (e.g., a server), or processor 221 may represent processing functionality of a plurality of devices operating in coordination (e.g., one or more servers, user interface devices 218, devices that are part of external resources 224, electronic storage 222, and/or other devices).

With continuing reference to FIG. 8B, the processor 221 is configured via machine-readable instructions to execute one or more computer program components. The computer program components may comprise one or more of information component 231, training component 232, prediction component 234, annotation component 236, trajectory component 238, and/or other components. Processor 221 may be configured to execute components 231, 232, 234, 236, and/or 238 by: software; hardware; firmware; some combination of software, hardware, and/or firmware; and/or other mechanisms for configuring processing capabilities on processor 221.

It should be appreciated that although components 231, 232, 234, 236, and 238 are illustrated as being co-located within a single processing unit, in embodiments in which processor 221 comprises multiple processing units, one or more of components 231, 232, 234, 236, and/or 238 may be located remotely from the other components. For example, in some embodiments, each of processor components 231, 232, 234, 236, and 238 may comprise a separate and distinct set of processors. The description of the functionality provided by the different components 231, 232, 234, 236, and/or 238 described below is for illustrative purposes, and is not intended to be limiting, as any of components 231, 232, 234, 236, and/or 238 may provide more or less functionality than is described. For example, one or more of components 231, 232, 234, 236, and/or 238 may be eliminated, and some or all of its functionality may be provided by other components 231, 232, 234, 236, and/or 238. As another example, processor 221 may be configured to execute one or more additional components that may perform some or all of the functionality attributed below to one of components 231, 232, 234, 236, and/or 238.

The disclosed approach relates to advanced imaging solutions and systems to augment camera images in real-time with clinically relevant information such as neural and bony structures. An output of system 25 may be a camera image that has overlaid in real-time relevant structures. The user may select what level of information/refinement may be required. The overlay may be performed based on confidence intervals of region of interest s of anatomical structures. The confidence intervals may be determined based on information available prior to access and then updated to tighten estimated regions of interest as new information becomes available intra-operation, e.g., as the camera is advanced in the port. FIG. 4B is an example output that can be displayed on a system monitor.

In some embodiments, the confidence interval may be similar to or the same as confidence interval described above. And each one may encode how confident the system 25 is that the anatomical structure is indeed what it is predicted to be. For example, annotation component 236 may overlay a transition zone or margin, or it may annotate a color-coded bullseye, where green indicates uppermost confidence. And, when annotating presence of Kambin's triangle 24 in a camera image, as the triangle's boundary extends outward it may become more red. The red portion may represent that, while still being near Kambin's triangle 24, there may be less confidence of that being the case. A same or similar approach may be performed when indicating a nerve root or other structure. For example, annotation component 236 may indicate where the center of the nerve is with 100% confidence; but the boundary may change in appearance, when extending outwards, indicating increased dubiousness.

In some embodiments, annotation component 236 may indicate each pixel of a captured image as to whether it represents a nerve, Kambin's triangle, or other structure(s). For example, prediction component 234 may indicate that there is a 90% probability that a pixel represents Kambin's triangle 24 and a 60% probability that the pixel represents nerve 21. In this example, annotation component 236 may then take a maximum of these two probabilities, when determining to annotate that pixel or region positively as Kambin's triangle. Alternatively, there may be a color code that blends colors (e.g., red and green) to visually represent a level of confidence that the prediction is accurate. Irrespective of this annotation approach, the representations may be updated in real-time upon obtaining access and when advancing camera 51 therein.

In some embodiments, models 264 may be a single convolutional neural network or another neural network that outputs all three of: (i) vertebral bodies and foramen, (ii) nerve roots, and (iii) bony landmarks. In other embodiments, there may be three networks, each of which outputting one of those three different types of anatomical structures. Accordingly, semantic segmentation is a contemplated approach. A class probability may be predicted for each structure of said three different types. For each pixel there may be a probability that the pixel is a particular anatomical structure. For instance, in one example the probability of a pixel can be 80% Kambin's triangle 24, 10% superior articular process (SAP), and 10% exiting nerve 21. Based on this prediction, the annotation would indicate that the pixel belongs to Kambin's triangle 24. The detections can be further enhanced by leveraging shape priors, for instance, pixels representing the Kambin's triangle can be grouped to resemble a triangle.

In some implementations, pre-op scans from CT 255 and/or Mill 256 may have been taken some time beforehand (e.g., a month), which may be significant as the region of interest may have already changed and/or the patient position during surgery may be different than from during the scans, rendering the scans somewhat outdated. And then, when a tool and/or medical practitioner accesses the scene, the region of interest becomes manipulated. Image(s) captured from camera 51 may thus provide an anchor that shows the actual real-time region of interest, as opposed to the CT and/or Mill scans that merely show what is expected at the region of interest.

In some embodiments, prediction component 234 may adapt predictions based on a patient, e.g., by predicting with just the CT scan and then adjusting the prediction or re-predicting based on images captured from camera 51 in real-time. As such, these images may be used together with previously taken scans of a patient. For example, the scan from CT 255 may help determine the region of interest; and then, when starting to use camera 51, a prediction of a location of Kambin's triangle 24 may be updated in real-time. In this or another example, the orientation may change. The pre-operative scans can provide additional information for the processor to identify Kambin's triangle 24 and/or to adjust the actual trajectory the advancing trocar 30.

In some embodiments, the trajectory component 238 may determine whether a trajectory of the advancement of the trocar 30 satisfies a criterion. The trajectory component 238 can then provide correction data to the trajectory such that the criterion is satisfied, in response to the determination that the trajectory did not satisfy the criterion. Thus, the processor can display correction information to adjust the actual trajectory of the trocar 30 to the desired trajectory of the trocar 30 that satisfies the criterion.

Although herein contemplated are embodiments that recognize or detect anatomical structures from only camera images, the CT and/or Mill scans help (e.g., with relative orientation and sizes) by providing more information that may be used to enhance accuracy of said recognition or detection. For example, nerve roots 21, 23 may be identified using an MRI scan that corresponds to a captured image, model 264 being trained with training data comprising ground truth labeled based on nerve root structures identified in previously-taken MRI scans.

In one example, the prediction component 234 may predict the presence of one or more anatomical structures (e.g., Kambin's triangle, nerves, soft tissue, and/or a bony structure) using only the camera 51 and a convolutional neural network or U-Net. It is recognized that a U-Net is a (deep) convolutional neural network for application in biomedical image segmentation. In another example, a prediction of anatomical structures may be performed using at least one pre-operation scan from at least one of MRI 256 and CT 255. In still another example with the navigated camera 51, the prediction may be performed using an output from a two-dimensional (2D) CT (e.g., C-Arm 254). The prediction component 234 may use 2D to 3D image reconstruction to identify Kambin's triangle and/or bony landmarks. Annotation component 236 may then overlay a representation of the identification(s) on the camera image as described above with respect to FIG. 4B. Accordingly, calibration targets or a navigated C-arm may be used to predict Kambin's triangle based on atlas or statistical shape models depending on patient phenotype.

In one or more of these embodiments, an expert or surgeon with prior knowledge may annotate or label images of training data beforehand (e.g., which surgeons already built indicating location of anatomical structures) and directly learn, e.g., from a statistical set of other samples to then build upon it. The annotated images may be with various levels of tissue penetration or bioavailability. Upon being trained, models 264 of these embodiments may be used to predict presence of these structures in captured images, each with corresponding confidence intervals around these structures. As more images are available during access, bounds of these structures may tighten.

In some embodiments, trajectory component 238 may use the camera images and various landmarks to provide orientation information and correction (e.g., when non-navigated). For example, if the camera orientation is changed during the medical procedure, this component may keep the same field of view by analyzing the rotation of landmarks of the image and maintaining a constant orientation of the image, which can be preferred by the user. As the camera turns, prediction component 234 may detect the structures somewhere else; then, trajectory component 238 may deduce how much camera 51 was turned to keep that pose. In embodiments where the camera is navigated, then trajectory component 238 may already know how much of the scene was rotated to perform a suitable correction.

In some embodiments, information component 231 may store information about how the region of interest was accessed to then learn from that (e.g., as model parameters or for hyperparameter tuning). In these or other embodiments, 3D CT scans may enable use of prior bony anatomy information to perform training of models 264. The convolutional neural network(s) implemented by models 264 may perform segmentation. This segmentation may be of spine structures via the CT scan. The segmentation helps with detection of these structures, e.g., when Kambin's triangle 24 is suspected to be below or above a particular structure. As such, context of what is in the image of camera 51 may be determined, increasing probability of an improved detection of said triangle.

In some embodiments, patient demographic information (e.g., size, weight, gender, bone health, or another attribute) and what level may be of the lumbar (e.g., L1, L2 versus L4, L5) may be obtained via training component 232 and/or prediction component 234. These attributes may serve as model parameters or in hyperparameter tuning, to help improve performance.

As mentioned, some embodiments of a convolutional neural network of model 264 may have as input (i.e., for training and when in deployment) just camera images, e.g., with surgeons performing the ground truth annotations. But other embodiments may have camera images, some high-level information, and CT scans (and even potentially further using MM scans) for said input, e.g., using the 3D CT scan segmentation results as ground truth. The overlaid outputs (e.g., as shown in FIG. 4B), from these embodiments, may be similar, but with the latter, other embodiments having more accurate predictions without requiring surgeons anymore for labeling due to having the segmentation information. In other words, the CT scan may be good enough to automatically detect, via a convolutional neural network, Kambin's triangle 24, to then transfer that learning to the convolutional neural network using camera images.

In some embodiments, annotations for the learning performed using CT scans may be supervised, unsupervised, or semi-supervised.

In some embodiments, the annotation component 236 may provide a medical practitioner with a user interface that indicates respective locations of anatomical structures (e.g., in the region displayed in the example of FIG. 4B or another region of interest) based on a set (e.g., one or more) of images from camera 51 taken in real-time during an approach to Kambin's triangle 24. In these or other embodiments, the trajectory component 238 can determine (e.g., for informing the medical practitioner) what changes need to be made in to the actual trajectory of the trocar 30, so as to achieve an improved trajectory towards Kambin's triangle 24. In either embodiments, the disclosed implementation of artificial intelligence may improve efficiency and safety of surgical equipment via improved accuracy in identifying Kambin's triangle 24. For example, needless and erroneous movements of a surgical instrument that cause contact with a nerve can be avoided.

In some embodiments, the trajectory component 238 may determine and continually update in real-time a working distance from the camera 51 (and thus from the trocar 30) to Kambin's triangle. In these or other embodiments, the trajectory component 238 may determine a position of a device such as the trocar 30 (including the first and second locations of the trocar 30) advancing towards Kambin's triangle. The image may be captured via at least one of the camera 51 and a charge coupled device (CCD) such as the electrical electrodes 52 of the neuro-monitoring system described above with respect to FIGS. 3A-3F).

In some embodiments, the annotation component 236 may indicate in near real-time at least one of Kambin's triangle 24, SAP 53, and nerve 21. If desired, the boundaries between anatomical structures may be indicated with thicker lines on the camera image, and text indicating each of these structures may be annotated thereon as well. Alternatively, pixels or other marks may be used to differentiate the structure. A user may, via user interface devices 218, select what should be emphasized and how such representative emphasis should be performed. For example, such user-configurable annotating may make the SAP boundary (or boundary of the nerve or Kambin's triangle) and/or corresponding text optional.

In some embodiments, trajectory component 238 may identify a way of advancing a tool towards or through Kambin's triangle 24, without touching a nerve, based on a relative location of SAP 53, superior endplate vertebrae, and/or another structure in the region of interest that may act as an anatomical landmark. From a CT scan, presence of SAP 53, levels of the spinal cord, and/or other bony landmarks may be predicted, each of which being predicted at a set of particular locations. And, from an MRI scan, nerve roots 21, 23 may be predicted as being present at particular locations. To identify Kambin's triangle 24, the presence of three edges may be predicted, e.g., including nerve 21, SAP 53, and the superior endplate vertebrae.

In some embodiments, the camera 51 may perform hyper-spectral or multi-spectral imaging (i.e., for wavelengths other than just white light) for visually obtaining information on blood supply, arteries, nerves, and the like. Overlaying information about these other wavelengths may also be optional for a user.

In some embodiments, the trajectory component 238 may identify the position of the trocar or dilators as they advance toward Kambin's triangle 24, and based on those images give the medical practitioner feedback. Herein, a medical practitioner may refer to human-based surgery, a combination of computer usage and human surgery, or pure automation.

As described above, the camera 51 can be mounted at the tip 37 of the trocar 30 (see FIGS. 3A-3D) that is configured to establish an access path toward or through Kambin's triangle. The camera 51 can be used to find or identify anatomical structures, including Kambin's triangle 24, one or more nerves, bony structure, and soft tissue. Thus, the camera 51 can be centrally located at the distal end of the trocar 30 on the central axis. In other examples, the camera can be disposed or embedded on a side wall of the trocar 30 between the exterior and interior surface.

In an implementation, certain access devices, such as trocars, can be inserted along a trajectory through Kambin's triangle 24 to the intervertebral disc space. The trocar can establish the trajectory through Kambin's triangle 24. In some examples, as described above, the access port 130 (see FIG. 5A) can be driven along the trajectory through Kambin's triangle to the intervertebral disc space. Surgical implements, such as surgical instruments and/or surgical implants, can thus be driven through the access port 130 in the manner described above. In other examples, other access devices, such as dilators and an access cannula, may be inserted to Kambin's triangle 24 along the trajectory. In some examples, the dilators and access cannula can be inserted to but not through Kambin's triangle 24. The access devices can define a working channel that extends to Kambin's triangle 24. Surgical instruments, such as a disc removal instrument, can be driven through the working channel and through Kambin's triangle to the intervertebral disc space to remove disc material from the disc space in preparation for insertion of an intervertebral implant.

In some embodiments, the camera 51 may be navigated (e.g., the CT scan and port/camera being registered). For example, navigation of the camera 51 may be tracked in real-time for a given known position in space of the trocar. That is, the position of the trocar may be registered to the pre-operation image such that the position of the anatomical structure is known relative to each other. As described above, the camera 51 may be on the side of the access device. This may be compensated for, knowing that the working channel is going to be then directionally offset from the camera (e.g., by a few millimeters). Further, the side wall of the trocar can be angled with respect to the actual trajectory of the trocar. Thus, the field of view can be oriented in a plane that is angularly offset with respect to a plane that is perpendicular to the central axis of the trocar and to the actual trajectory of the trocar as described above. In one example, the angle be about 30 degrees. Alternatively, the camera 51 can be mounted on the angled side wall and oriented parallel to the central axis of the trocar 30.

It is recognized that there can be some distortion that may be corrected based on the known angle and at least an approximation of the offset distance from the central axis. Accordingly, the image that is provided from camera 51 may be based on the camera location being skewed so that the center of the image is aligned with the central axis of the trocar, and software corrections may be performed via an imaging processing pipeline. In some implementations, when looking from the side, a user may get a lot more distortion on the top, the further away the camera 51 is, with the angle. But a majority of this may be corrected. When corrected, an entirety of the image can appear centered to the user. When not corrected, there may be a lens effect due to being off the center. This may not cause a loss of information, there being rather just different pixels representing a different area. This is known as fisheye distortion and may be corrected.

In implementations comprising CT 255, a patient may be laying on a table when scanned. Then, at a different time which can be a number or days or weeks later, at the date of surgery, the patient may be laying at a different position when inserting the camera 51 into the surgical site in the manner described above. For example, a radio-opaque marker may be placed nearby and fastened to the lumbar region (e.g., L5-S1 area). A CT scan may be performed, and then the inserted marker can be identified in the CT scan. Accordingly, the patient, images, or devices may be registered, e.g., by aligning the marker on the image from the camera 51 with the coordinate system from the previous CT scans. The registration may further be performed with a scanner and the reference array 40 as described above (see FIG. 3A). The flexible nature of the spine can increase the risk of movement and thereby inaccuracies, making navigation significant for improving accuracy.

In embodiments where the camera 51 is navigated and when 3D CT scans are used, prediction component 234 may automatically segment the CT scan (e.g., using deep learning) to identify vertebral bodies and foramen; a foramen is an open hole that exists in the body of animals to allow muscles, nerves, arteries, veins, or other structures to connect one part of the body with another. From these identifications, the prediction component 234 may deduce Kambin's triangle 24. A representation of Kambin's triangle may then be overlaid on the image of the camera 51 as shown at FIG. 4B. In these or other embodiments, the prediction component 234 may automatically segment the CT scan (e.g., using deep learning trained through co-acquired Mill CT scans) to identify exiting nerve 21. Annotation component 236 may then overlay this neural structure on the image. In these or other embodiments, prediction component 34 may automatically segment the CT scan (e.g., using deep learning) to identify such bony landmarks as the vertebra, pedicle, transverse process (TP), spinous process (SP), and/or SAP 53. Then, the annotation component 236 may overlay the bony structures on the camera's image. As such, the annotation component 236 may simultaneously overlay at least one of Kambin's triangle 24, the neural structures 21,23, and the bony structures on the image, with options for the user to refine an amount of information displayed.

In some embodiments, a machine learning model may be inputted 3D scans to predict where Kambin's triangle 24 is in each one (via supervised or unsupervised learning); and then another machine learning model may be trained using labels based on these predictions such that this other model makes predictions of Kambin's triangle using an image of a 2D camera. In other embodiments, human labeling of Kambin's triangle may be used for training a machine learning model; and then both the 2D camera and the 3D scans may be input into this model for predicting said triangle in real-time of a current patient. These embodiments implement distillation learning or student-teacher models.

In some embodiments, the camera 51 may be non-navigated, even though 3D CT scans may be available. For example, deep learning may be performed to identify various bony landmarks directly from the 2D camera image as the intra-operation images of camera 51 are fed into this prediction model in real-time. In other embodiments, a user may have to wait some time (e.g., 10 seconds) to obtain a prediction of an identification of Kambin's triangle 24; nothing may move during that time period. The predictions may not be as fast as the camera feed itself, in some implementations, but it can update itself in near real-time as things move. For example, the port may be rotated (or a tool moved) to look from a particular angle.

When not navigated, a registration with a 3D CT scan may be performed in real-time based on landmarks that are found. Then, a confidence interval of Kambin's triangle, neural structures, and bony structures may be overlaid on the camera image. Due to the registration not being performed, a user may not know where the camera is looking versus where the CT scanner is looking. When navigated and registered, the user would know exactly where a 2D slice of the camera image is looking within the 3D CT scan. When not using a navigated camera, a user may know how a patient's bony anatomy looks, but they would have no way to link that to the camera image. This non-navigated approach may thus involve obtaining a prediction of the bones from the camera image and then registering that back to the 3D CT scan, which can be used to also predict presence of the bones to then estimate which 2D slice at which the user is looking.

In some embodiments, CT 255 is an XT (cone beam CT). In other embodiments where there is no CT scan available, then prediction component 34 can rely on some visual indicia. In other examples, a nerve locator device or some other means, such as an ultrasound or Sentio™ mechanomyographic (MMG) system, can be used to locate and map the nerves using electrical stimulus of the nerves, thereby providing further imaging input to overlay on the camera image. The Sentio™ MMG system can be located in the distal region 36, and in particular in the second portion 36b of the distal region 36 (see FIG. 3A) to control the trajectory of the trocar 30. In an example, an integrated device may be used to send an electric current through a probe or port to the electrodes 52 (see FIGS. 3A-3E) to determine the distance to a nerve or other anatomy of interest in the manner described above.

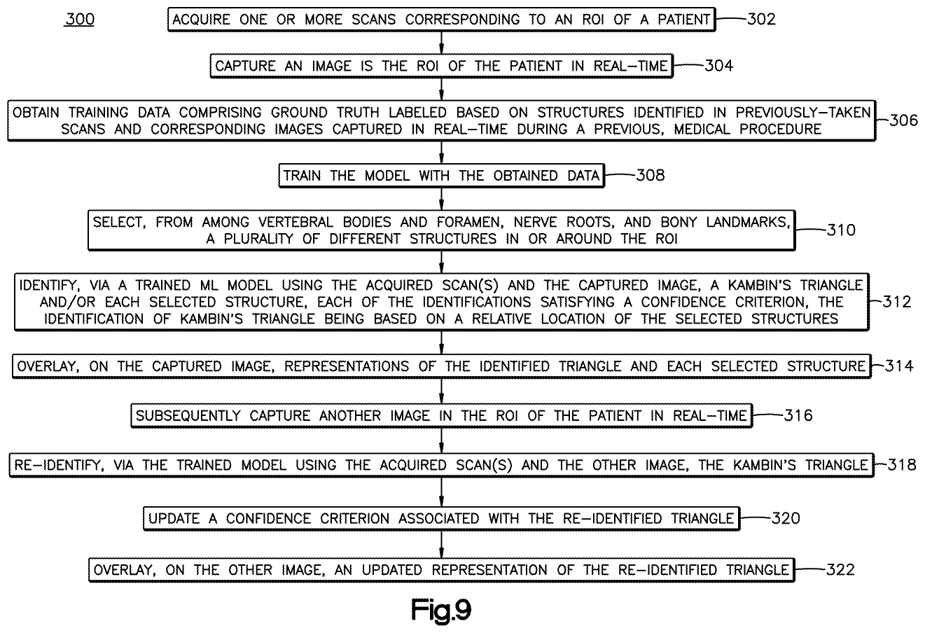

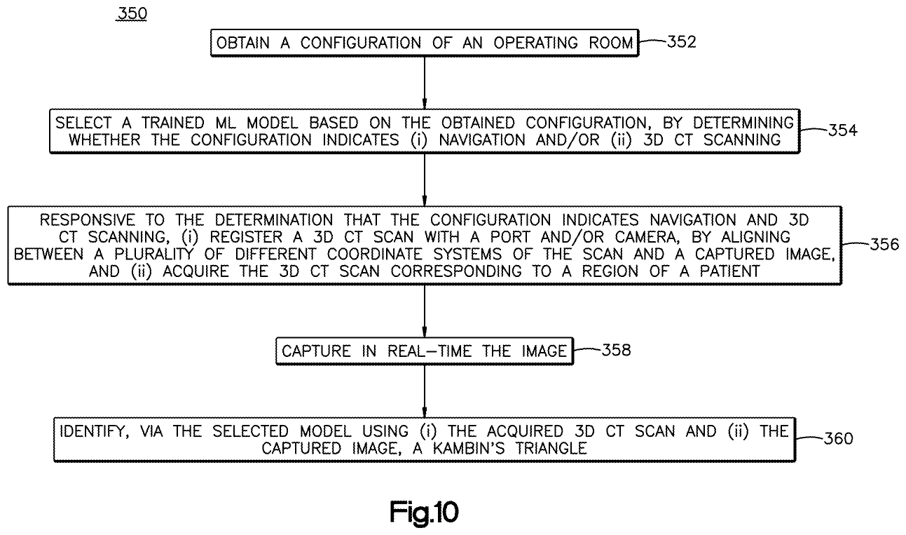

FIGS. 9-10 illustrate methods 300 and 350 for conducting accurate surgery with enhanced imaging, in accordance with one or more embodiments. These methods may be performed with a computer system comprising one or more computer processors and/or other components. The processors are configured by machine readable instructions to execute computer program components. The operations of these methods, which are presented below, are intended to be illustrative. In some embodiments, methods 300 and 350 may each be accomplished with one or more additional operations not described, and/or without one or more of the operations discussed. Additionally, the order in which the operations of each of these methods are illustrated in FIGS. 9-10. In some embodiments, each of methods 300 and 350 may be implemented in one or more processing devices (e.g., a digital processor, an analog processor, a digital circuit designed to process information, an analog circuit designed to process information, a state machine, and/or other mechanisms for electronically processing information). The processing devices may include one or more devices executing some or all of the operations of these methods in response to instructions stored electronically on an electronic storage medium. The processing devices may include one or more devices configured through hardware, firmware, and/or software to be specifically designed for execution of one or more of these operations.

At operation 302 of method 300, one or more scans corresponding to an region of interest of a patient may be acquired. For example, obtained patient scans may be unsegmented and correspond to a surgical region or a planned surgical region. In some embodiments, operation 302 is performed by a processor component the same as or similar to information component 231 and C-Arm 254, CT 255, and/or MRI 256 (shown in FIG. 8B and described herein).

At operation 304 of method 300, an image in the region of interest of the patient may be captured in real-time. For example, camera 51 may take a set of images internal to the body or patient in real-time, the capturing of the set of images being performed during the procedure. Method 300 may be executed using one or more images at a time. In some embodiments, operation 304 is performed by a processor component the same as or similar to information component 231 and camera 51.

At operation 306 of method 300, training data may be obtained, the training data comprising ground truth labeled based on structures identified in previously-taken scans and corresponding images captured in real-time during a previous, medical procedure. In some embodiments, operation 306 is performed by a processor component the same as or similar to the training component 232 of FIG. 8B.

At operation 308 of method 300, the model may be trained with the obtained training data. For example, a trained convolutional neural network or another of models 264 may be obtained for performing recognition or detection of anatomical structures in the images and/or scans. That is, after training component 232 trains the neural networks, the resulting trained models may be stored in models 264 of prediction database 260. In some embodiments, operation 308 is performed by a processor component the same as or similar to training component 232.

At operation 310 of method 300, a plurality of different structures in, near, and/or around the region of interest may be selected (e.g., manually via user interface devices 218 or automatically based on a predetermined configuration) from among vertebral bodies and foramen, nerve roots, and bony landmarks. In some embodiments, operation 310 is performed by a processor component the same as or similar to information component 231 (shown in FIG. 8B and described herein).

At operation 312 of method 300, a Kambin's triangle and/or each selected structure may be identified, via a trained machine learning (ML) model using the acquired scan(s) and the captured image; each of the identifications may satisfy a confidence criterion, the identification of Kambin's triangle being based on a relative location of the selected structures. For example, the predicting is performed by identifying presence of at least one neurological structure from the unsegmented scan using an image analysis tool that receives as an input the unsegmented scan and outputs a labeled image volume identifying the at least one neurological structure. In some embodiments, prediction component 234 may predict via a U-Net, which may comprise a convolutional neural network developed for biomedical image segmentation and/or a fully convolutional network. In some embodiments, operation 312 is performed by a processor component the same as or similar to prediction component 234 (shown in FIG. 8B and described herein).

At operation 314 of method 300, representations of the identified triangle and/or of each selected structure may be overlaid, on the captured image. For example, information distinguishing, emphasizing, highlighting, or otherwise indicating anatomical structures may overlay the images, on a path of approach to Kambin's triangle 24. In some embodiments, operation 314 is performed by a processor component the same as or similar to annotation component 236 (shown in FIG. 8B and described herein).

At operation 316 of method 300, another image in the region of interest of the patient may be subsequently captured in real-time. In some embodiments, operation 316 is performed by a processor component the same as or similar to information component 231 and camera 51.

At operation 318 of method 300, the Kambin's triangle may be re-identified, via the trained model using the acquired scan(s) and the other image. For example, a subsequent identification of Kambin's triangle 24 may satisfy an improved confidence criterion, e.g., for growing a region that represents the identified triangle based on a subsequently captured image. In some embodiments, operation 318 is performed by a processor component the same as or similar to prediction component 234.

At operation 320 of method 300, a confidence criterion associated with the re-identified triangle may be updated. For example, a confidence interval may be updated in real-time based on a feed of camera 51. In some embodiments, annotation component 236 may determine a confidence interval, e.g., while camera 51 is in proximity to the region of interest and/or Kambin's triangle 24. The confidence interval may indicate an extent to which an anatomical structure is predicted to be present at each of a set of locations (e.g., 2D, 3D, or another suitable number dimensions). In some embodiments, a confidence criterion may be satisfied by an extent that improves upon known means, a higher assurance being obtained that Kambin's triangle 24 is indeed at the predicted location (e.g., for advancing towards said triangle). In some embodiments, operation 320 is performed by a processor component the same as or similar to prediction component 234 or annotation component 236.

At operation 322 of method 300, an updated representation of the re-identified triangle may be overlaid, on the other image. For example, the overlaying may be on a same or different image from the one used to make the prediction. In some embodiments, operation 322 is performed by a processor component the same as or similar to annotation component 236.

At operation 352 of method 350 as depicted in FIG. 10, a configuration of an operating room may be obtained. For example, whether an output from a 2D CT, 3D CT, and an MM are available may be determined. In this or another example, whether camera 51 is navigable or registrable may be determined. In implementations where only CT scans are available of a patient, training component 232 may use Mill scans of other patients to train a method that can detect the nerves from the CT scan, as discussed herein. As such, by knowing where certain bony structures or landmarks are, prediction component 234 may predict where the nerve is. The algorithmic approach may itself be determined based on availability of specific tools and technologies. For example, system 25 may thus use certain inputs and/or conditions and then adjust itself to pick a suitable method. In some embodiments, operation 352 is performed by a processor component the same as or similar to information component 231.

At operation 354 of method 350, a trained machine-learning model may be selected based on the obtained configuration, by determining whether the configuration indicates navigation and/or 3D CT scanning. In some embodiments, operation 354 is performed by a processor component the same as or similar to training component 232 or prediction component 234.

At operation 356 of method 350, responsive to the determination that the configuration indicates navigation and 3D CT scanning, a 3D CT scan may be registered with a port and/or camera (e.g., by aligning between a plurality of different coordinate systems and a captured image), and the 3D CT scan corresponding to a region of a patient may be acquired. In some embodiments, operation 356 is performed by a processor component the same as or similar to trajectory component 238 (shown in FIG. 8B and described herein).

At operation 358 of method 350, the image may be captured in real-time.

At operation 360 of method 350, Kambin's triangle may be identified, via the selected model using the acquired 3D CT scan and the captured image. In some embodiments, operation 360 is performed by a processor component the same as or similar to prediction component 234.

Techniques described herein can be implemented in digital electronic circuitry, or in computer hardware, firmware, software, or in combinations of them. The techniques can be implemented as a computer program product, i.e., a computer program tangibly embodied in an information carrier, e.g., in a machine-readable storage device, in machine-readable storage medium, in a computer-readable storage device or, in computer-readable storage medium for execution by, or to control the operation of, data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network.

Method steps of the techniques can be performed by one or more programmable processors executing a computer program to perform functions of the techniques by operating on input data and generating output. Method steps can also be performed by, and apparatus of the techniques can be implemented as, special purpose logic circuitry, e.g., an FPGA (field programmable gate array) or an ASIC (application-specific integrated circuit). FIGS. 8B-10 are further described in U.S. patent application Ser. No. 17/390,115 filed Jul. 30, 2021, the disclosure of which is hereby incorporated by reference as if set forth in its entirety herein.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, such as, magnetic, magneto-optical disks, or optical disks. Information carriers suitable for embodying computer program instructions and data include all forms of non-volatile memory, including by way of example semiconductor memory devices, such as, EPROM, EEPROM, and flash memory devices; magnetic disks, such as, internal hard disks or removable disks; magneto-optical disks; and CD-ROM and DVD-ROM disks. The processor and the memory can be supplemented by, or incorporated in special purpose logic circuitry. Disclosure of communication of a component with a processor can include direct data communication with the processor or indirect data communication with the processor. In one example, indirect communication with the processor can include communication with memory for storage in memory that, in turn, is in direct communication with the processor such that the processor can retrieve the data stored in the memory.

Several embodiments of the present disclosure are specifically illustrated and/or described herein. However, it will be appreciated that modifications and variations are contemplated and within the scope of the appended claims.

The disclosure of U.S. Pat. No. 8,518,087 is hereby incorporated by reference in its entirety and should be considered a part of this specification.

It should be appreciated that the illustrations and discussions of the embodiments shown in the figures are for exemplary purposes only and should not be construed limiting the disclosure. One skilled in the art will appreciate that the present disclosure contemplates various embodiments. Additionally, it should be understood that the concepts described above with the above-described embodiments may be employed alone or in combination with any of the other embodiments described above. It should be further appreciated that the various alternative embodiments described above with respect to one illustrated embodiment can apply to all embodiments as described herein, unless otherwise indicated.

What is claimed is:

1. A surgical system, comprising:
a trocar having a trocar body and first and second sensors supported by the trocar body, wherein each of the first and second sensors is configured to sense at least one of a position of the trocar, an orientation of the trocar, a property of tissue proximate to the trocar, and a distance from a tissue and the trocar;
a display; and
a processor in communication with the plurality of sensors and the display,
wherein the processor is configured to overlay on the display graphical representations of data from each of the sensors as the trocar is advanced toward a target anatomical site.

2. The system of claim 1, wherein one of the plurality of sensors comprises a visible light image sensor.

3. The system of claim 2, wherein the graphical representations of data from each of the plurality of sensors are highlighted regions of an image captured by the visible light image sensor denoting the presence of any of a type of tissue and an anatomical structure.

4. The system of claim 3, wherein the anatomical structure includes any of bone and nerve.

5. The system of claim 1, wherein one of the plurality of sensors is configured to sense a position of the trocar and the processor is configured to provide on the display a graphical indication of alignment between an actual trajectory of the trocar and a desired trajectory of the trocar.

6. The system of claim 5, wherein the graphical indication of alignment between the position of the trocar and a desired insertion trajectory includes a first icon representing a position of a first location of the trocar and a second icon representing a position of a second location of the trocar.

7. The system of claim 1, wherein one of the plurality of sensors comprises a nerve mapping electrode.

8. The system of claim 7, wherein the plurality of nerve mapping electrodes are spaced around a distal region of the trocar and the processor is configured to triangulate nerve location based on any of time-of-flight and signal strength detection at two or more of the plurality of nerve mapping electrodes.

9. The system of claim 7, wherein the plurality of nerve mapping electrodes are embedded in the transparent distal tip of the trocar.

10. The system of claim 1, wherein one of the plurality of sensors comprises a non-visible light image sensor, and the processor is further configured to differentiate tissue type based on images captured by the non-visible light image sensor.

11. The system of claim 1, wherein the trocar is configured to deliver haptic feedback to a user based on input from the plurality of sensors.

12. The system of claim 1, further comprising a flexible access port, wherein the trocar extends through a lumen of the surgical access port.

13. A surgical system, comprising:
a trocar having trocar body and a camera supported by the trocar body;
a display; and
a processor in communication with the camera and the display,
wherein the processor is configured to overlay on the display a pre-operative image of anatomy onto a real-time image of the anatomy from the camera as the trocar travels toward a target anatomical site.

14. The system of claim 13, wherein the anatomy includes any of bone and nerve.

15. The surgical system of claim 13, wherein the trocar has a transparent tip, and the camera views the anatomy distal of the trocar through the transparent tip.

16. The system of claim 13, further comprising an electrode supported by the trocar body and configured to apply an electrical current to a nerve so as to determine a distance between the trocar and the nerve.

17. The system of claim 13, further comprising a position sensor configured to detect a position of the trocar, wherein the processor is configured to provide on the display a graphical indication of alignment between the position of the trocar and a desired insertion trajectory.

18. The system of claim 17, wherein the graphical indication of alignment between the position of the trocar and a desired insertion trajectory includes a first icon representing a position of a first location of the trocar and a second icon representing a position of a second location of the trocar, wherein the first location is spaced from the second location in a distal direction.

19. The system of claim 13, further comprising a flexible access port having a lumen, wherein the trocar is received in the lumen.

20. The system of claim 13, wherein the camera comprises a non-visible light image sensor.
